(19) United States
(12) Patent Application Publication
(10) Pub. No.: US 2006/0258571 A1
(43) Pub. Date: Nov. 16, 2006

(54) Antibacterial ester macrocycles

(76) Inventors: Thomas Lampe, Dusseldorf (DE); Isabelle Adelt, Dusseldorf (DE); Dieter Beyer, Wuppertal (DE); Nina Brunner, Essen (DE); Rainer Endermann, Wuppertal (DE); Kerstin Ehlert, Velbert (DE); Hein-Peter Kroll, Wuppertal (DE); Franz Von Nussbaum, Dusseldorf (DE); Siegfried Raddatz, Koln (DE); Joachim Rudolph, Guilford, CT (US); Guido Schiffer, Wuppertal (DE); Andreas Schumacher, Efringen-Kirchen (DE); Yolanda Cancho-Grande, Hilden (DE); Martin Michels, Leverkusen (DE); Stefan Weigand, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/522,667

(22) PCT Filed: Jul. 18, 2003
(86) PCT No.: PCT/EP03/07824

§ 371 Date: Jul. 27, 2006

(30) Foreign Application Priority Data
Jul. 29, 2002 (JP) 102 34 422.1

(51) Int. Cl. A61K 38/12; A61K 31/395; C07K 5/12

(52) U.S. Cl. 514/9; 514/183; 530/317; 540/460

(57) ABSTRACT

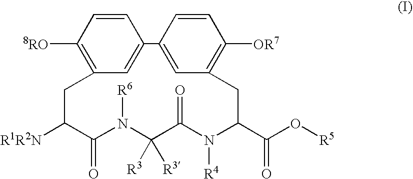

The invention relates to antibacterial ester macrocycles of formula (I), methods for the production thereof, and the use thereof for producing medicaments used for the treatment and/or prophylaxis of diseases, particularly bacterial infections.

The invention relates to antibacterial ester macrocycles and processes for their preparation, and to their use for producing medicaments for the treatment and/or prophylaxis of diseases, in particular of bacterial infections.

U.S. Pat. No. 3,452,136, thesis of R. U. Meyer, Stuttgart University, Germany 1991, thesis of V. Leitenberger, Stuttgart University, Germany 1991, Synthesis (1992), (10), 1025-30, J. Chem. Soc., Perkin Trans. 1 (1992), (1), 123-30, J. Chem. Soc., Chem. Commun. (1991), (10), 744, Synthesis (1991), (5), 409-13, J. Chem. Soc., Chem. Commun. (1991), (5), 275-7, J. Antibiot. (1985), 38(11), 1462-8, J. Antibiot. (1985), 38(11), 1453-61, describe the natural product biphenomycin B (R¹, R² are hydrogen, R^{3′}, R⁴, R⁷, R⁸ and R⁹ are hydrogen, R³ is 3-amino-2-hydroxy-prop-1-yl and free carboxyl instead of an ester group) as having antibacterial activity. Some steps in the synthesis of biphenomycin B are described in Synlett (2003), 4, 522-525.

Chirality (1995), 7(4), 181-92, J. Antibiot. (1991), 44(6), 674-7, J. Am. Chem. Soc. (1989), 111(19), 7323-7, J. Am. Chem. Soc. (1.989), 111(19), 7328-33, J. Org. Chem. (1987), 52(24), 5435-7, Anal. Biochem. (1987), 165(1), 108-13, J. Org. Chem. (1985), 50(8), 1341-2, J. Antibiot. (1993), 46(3), C-2, J. Antibiot. (1993), 46(1), 135-40, Synthesis (1992), (12), 1248-54, Appl. Environ. Microbiol. (1992), 58(12), 3879-8, J. Chem. Soc., Chem. Commun. (1992), (13), 951-3 describe a structurally related natural product, biphenomycin A, which has a further substitution with a hydroxy group on the macrocycle.

The natural products do not in terms of their properties comply with the requirements for antibacterial medicaments. Although structurally different agents with antibacterial activity are available on the market, the development of resistance is a regular possibility. Novel agents for good and more effective therapy are therefore desirable.

One object of the present invention is therefore to provide novel and alternative compounds with the same or improved antibacterial effect for the treatment of bacterial diseases in humans and animals.

It has surprisingly been found that derivatives of these natural products in which the carboxyl group of the natural product is replaced by an ester group have antibacterial activity.

The invention relates to compounds of the formula 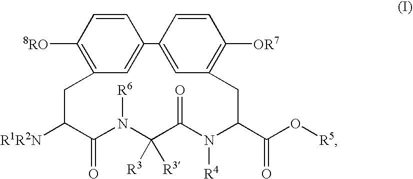
in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
   where R¹ apart from hydrogen may be substituted by 0, 1, 2 or 3 substituents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy and carboxyl,
R² is hydrogen or alkyl,
   where alkyl may be substituted by 0, 1, 2 or 3 substituents R²⁻¹, where the substituents R²⁻¹ are selected independently of one another from the group consisting of halogen, amino, alkylamino and dialkylamino, or
R¹ and R² together with the nitrogen atom to which they are bonded form a heterocycle which may be substituted by 0, 1 or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
   and in which one or more free amino groups in the side group of the amino acid may be substituted by alkyl, alkenyl, alkynyl, cycloalkyl, aryl, heteroaryl, heterocyclyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is alkyl, cycloalkyl, aryl, heteroaryl, heterocyclyl or a hydroxy function-linked amino acid residue, where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
   in which alkylamino and dialkylamino may be substituted by 0, 1, 2 or 3 substituents R⁵⁻², where the substituents R⁵⁻² are selected independently of one another from the group consisting of hydroxy, amino, alkoxy, alkylamino and dialkylamino,
R⁶ is hydrogen, C₁-C₆-alkyl or C₃-C₈-cycloalkyl,
R⁷ is hydrogen, C₁-C₆-alkyl, alkylcarbonyl or C₃-C₈-cycloalkyl,
R⁸ is hydrogen or C₁-C₆-alkyl,
   and their salts, their solvates and the solvates of their salts.

Compounds of the invention are the compounds of the formula (I) and the salts, solvates and solvates of the salts thereof, the compounds which are encompassed by formula (I) and are of the formula (I′) mentioned below, and the salts, solvates, and solvates of the salts thereof, and the compounds which are encompassed by formula (I) and/or (I′) and are mentioned below as exemplary embodiment(s), and the salts, solvates and solvates of the salts thereof, where the compounds which are encompassed by formula (I) and/or (I′) and are mentioned below are not already salts, solvates and solvates of the salts.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates refer for the purposes of the invention to those forms of the compounds which form a complex in the solid or liquid state by coordination with solvent molecules. Hydrates are a special form of solvates in which the coordination takes place with water.

For the purposes of the present invention, the substituents have the following meaning, unless specified otherwise:

Alkyl and the alkyl moieties in substituents such as alkoxy, mono- and dialkylamino, alkylsulfonyl include linear and branched alkyl, e.g. C₁-C₁₂-, in particular C₁-C₆- and C₁-C₄-alkyl.

C₁-C₆-Alkyl includes methyl, ethyl, n- and i-propyl, n-, i-, sec- and tert-butyl, n-pentyl, isopentyl, neopentyl and hexyl,

C₁-C₄-Alkyl includes methyl, ethyl, n- and i-propyl, n-, i-, sec- and tert-butyl,

Alkylcarbonyl is for the purposes of the invention preferably a straight-chain or branched alkyl radical having 1 to 6 or 1 to 4 carbon atoms. Those which may be mentioned by way of example and preferably are: methylcarbonyl, ethylcarbonyl, n-propylcarbonyl, isopropylcarbonyl and t-butylcarbonyl.

Alkenyl includes linear and branched C₂-C₁₂-, in particular C₂-C₆- and C₂-C₄-alkenyl, such as, for example, vinyl, allyl, prop-1-en-1-yl, isopropenyl, but-1-enyl, but-2-enyl, buta-1.2-dienyl and buta-1.3-dienyl.

Alkynyl includes linear and branched C₂-C₁₂-, in particular C₂-C₆- and C₂-C₄-alkynyl, such as, for example, ethynyl, propargyl (2-propynyl), 1-propynyl, but-1-ynyl, but-2-ynyl.

Cycloalkyl includes polycyclic saturated hydrocarbon radicals having up to 14 carbon atoms, namely monocyclic C₃-C₁₂-, preferably C₃-C₈-alkyl, in particular C₃-C₆-alkyl such as, for example, cyclopropyl, cyclobutyl, cyclopentyl, cyclohexyl, cycloheptyl, cyclooctyl, cyclononyl, and polycyclic alkyl, i.e, preferably bicyclic and tricyclic, optionally spirocyclic C₇-C₁₄-alkyl, such as, for example, bicyclo[2.2.1]-hept-1-yl, bicyclo[2.2.1]-hept-2-yl, bicyclo[2.2.1]-hept-7-yl, bicyclo[2.2.2]-oct-2-yl, bicyclo[3.2.1]-oct-2-yl, bicyclo[3.2.2]-non-2-yl and adamantyl.

Aryl is for the purposes of the invention an aromatic radical preferably having 6 to 10 carbon atoms. Preferred aryl radicals are phenyl and naphthyl.

Alkoxy is for the purposes of the invention preferably a straight-chain or branched alkoxy radical in particular having 1 to 6, 1 to 4 or 1 to 3 carbon atoms. A straight-chain or branched alkoxy radical having 1 to 3 carbon atoms is preferreof theoryose which may be mentioned by way of example and preferably are: methoxy, ethoxy, n-propoxy, isopropoxy, tert-butoxy, n-pentoxy and n-hexoxy.

Alkoxycarbonyl is for the purposes of the invention preferably a straight-chain or branched alkoxy radical having 1 to 6 or 1 to 4 carbon atoms, which is linked via a carbonyl group. A straight-chain or branched alkoxycarbonyl radical having 1 to 4 carbon atoms is preferreof theoryose which may be mentioned by way of example and preferably are: methoxycarbonyl, ethoxycarbonyl, n-propoxycarbonyl, isopropoxycarbonyl and tert-butoxycarbonyl.

Monoalkylamino (alkylamino) is for the purposes of the invention an amino group having one straight-chain or branched alkyl substituent which preferably has 1 to 6, 1 to 4 or 1 or 2 carbon atoms. A straight-chain or branched monoalkylamino radical having 1 to 4 carbon atoms is preferreof theoryose which may be mentioned by way of example and preferably are: methylamino, ethylamino, n-propylamino, isopropylamino, tert-butylamino, n-pentylamino and n-hexylamino.

Dialkylamino is for the purposes of the invention an amino group having two identical or different straight-chain or branched alkyl substituents, which preferably each have 1 to 6, 1 to 4 or 1 or 2 carbon atoms. Straight-chain or branched dialkylamino radicals having in each case 1, 2, 3 or 4 carbon atoms per alkyl substituent are preferreof theoryose which may be mentioned by way of example and preferably are: N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Monoalkylaminocarbonyl (alkylaminocarbonyl) or dialkylaminocarbonyl is for the purposes of the invention an amino group which is linked via a carbonyl group and which has one straight-chain or branched or two identical or different straight-chain or branched alkyl substituents each preferably having 1 to 4 or 1 or 2 carbon atoms. Those which may be mentioned by way of example and preferably are: methylaminocarbonyl, ethylaminocarbonyl, isopropylaminocarbonyl, t-butylaminocarbonyl, N,N-dimethylaminocarbonyl, N,N-diethylaminocarbonyl, N-ethyl-N-methylaminocarbonyl and N-t-butyl-N-methylaminocarbonyl.

Arylaminocarbonyl is for the purposes of the invention an aromatic radical having preferably 6 to 10 carbon atoms, which is linked via an aminocarbonyl group. Preferred radicals are phenylaminocarbonyl and naphthylaminocarbonyl.

Alkylcarbonylamino (acylamino) is for the purposes of the invention an amino group having a straight-chain or branched alkanoyl substituent which preferably has 1 to 6, 1 to 4 or 1 or 2 carbon atoms and is linked via the carbonyl group. A monoacylamino radical having 1 or 2 carbon atoms is preferreof theoryose which may be mentioned by way of example and preferably are: formamido, acetamido, propionamido, n-butyramido and pivaloylamido.

Heterocyclyl (heterocycle) is a mono- or polycyclic, heterocyclic radical having 4 to 10 ring atoms and up to 3, preferably 1, heteroatoms or heterogroups from the series N, O, S, SO, SO₂. 4- to 8-membered, in particular 5- to 6-membered heterocyclyl is preferred. Mono- or bicyclic heterocyclyl is preferred. Monocyclic heterocyclyl is particularly preferred. N and O are preferred as heteroatoms. The heterocyclyl radicals may be saturated or partially unsaturated. Saturated heterocyclyl radicals are preferred. The heterocyclyl radicals may be linked via a carbon atom or a heteroatom. 5- to 6-membered, monocyclic saturated heterocyclyl radicals having up to two heteroatoms from the series O, N and S are particularly preferreof theoryose which may be mentioned by way of example and preferably are: oxetan-3-yl, pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, perhydroazepinyl, piperazin-1-yl, piperazin-2-yl. A nitrogen heterocyclyl ring is in this connection a heterocycle which has only nitrogen atoms as heteroatoms.

Heteroaryl is an aromatic, mono- or bicyclic radical having 5 to 10 ring atoms and up to 5 heteroatoms from the series S, O and/or N. 5- to 6-membered heteroaryls having up to 4 heteroatoms are preferred. The heteroaryl radical may be linked via a carbon atom or heteroatom. Those which may be mentioned by way of example and preferably are: thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Alkoxycarbonylamino is for the purposes of the invention an amino group having a straight-chain or branched alkoxycarbonyl substituent which preferably has 1 to 6 or 1 to 4 carbon atoms in the alkoxy radical and is linked via the carbonyl group. An alkoxycarbonylamino radical having 1 to 4 carbon atoms is preferreof theoryose which may be mentioned by way of example and preferably are: methoxycarbonylamino, ethoxycarbonylamino, n-propoxycarbonylamino and t-butoxycarbonylamino.

Carbonyl is a —C(O) group. Correspondingly, arylcarbonyl, heterocyclylcarbonyl and heteroarylcarbonyl are substituted on the carbonyl group by the appropriate radicals, i.e. aryl, heterocyclyl etc.

Sulfonyl is an —S(O)₂ group. Correspondingly, alkylsulfonyl, arylsulfonyl, heterocyclylsulfonyl and heteroarylsulfonyl are substituted on the sulfonyl group by the appropriate radicals, i.e. alkyl, aryl etc.

Aminosulfonyl is an —S(O)₂NH₂ group. Correspondingly, alkylaminosulfonyl, dialkylaminosulfonyl, arylaminosulfonyl, heterocyclylaminosulfonyl and heteroarylaminosulfonyl are substituted on the amino group by the appropriate radicals, i.e. alkyl, aryl etc.

Halogen includes for the purposes of the invention fluorine, chlorine, bromine and iodine. Fluorine or chlorine are preferred.

The side group of an amino acid means for the purposes of the invention the organic radical of an α-amino acid molecule which is linked to the α-carbon atom of the amino acid. Preference is given in this connection to the residues of naturally occurring α-amino acids in the L or in the D configuration, especially naturally occurring α-amino acids in the natural L configuration.

These include for example hydrogen (glycine), methyl (alanine), prop-2-yl (valine), 2-methylprop-1-yl (leucine), 1-methylprop-1-yl (isoleucine), a (3-indolyl)methyl group (tryptophan), a benzyl group (phenylalanine), a methylthioethyl group (methionine), hydroxymethyl (serine), p-hydroxybenzyl (tyrosine), 1-hydroxyeth-1-yl (threonine), mercaptomethyl (cysteine), carbamoylmethyl (asparagine), carbamoylethyl (glutamine), carboxymethyl (aspartic acid), carboxyethyl (glutamic acid), 4-aminobut-1-yl (lysine), 3-guanidinoprop-1-yl (arginine), imidazol-4-ylmethyl (histidine), 3-ureidoprop-1-yl (citrulline), mercaptoethyl (homocysteine), hydroxyethyl (homoserine), 4-amino-3-hydroxybut-1-yl (hydroxylysine), 3-aminoprop-1-yl (ornithine), 2-hydroxy-3-aminoprop-1-yl (hydroxyornithine).

Carbonyl-linked amino acid residue is an amino acid residue which is linked via the carbonyl group of the amino acid acidic function. Preference is given in this connection to α-amino acids in the L or in the D configuration, especially naturally occurring α-amino acids in the natural L configuration, e.g. glycine, L-alanine and L-proline.

Hydroxy function-linked amino acid residue is an amino acid residue which is linked via a hydroxy function of the amino acid. These include for exampler serine (—OCH(NH₂)COOH) or threonine (—OCH(CH₃)CH(NH₂)COOH. Preference is given in this connection to α-amino acids in the L or in the D configuration, especially naturally occurring α-amino acids in the natural L configuration, e.g. serine or threonine.

Amino protective groups means for the purposes of the present invention those organic radicals with which amino groups can be protected temporarily from attack by reagents, so that reactions such as oxidation, reduction, substitution and condensation take place only at the desired (unprotected) sites. They are stable for the duration of the protection under all conditions of the reactions and purification operations to be carried out and can be eliminated again selectively and with high yield under mild conditions (Römpp Lexikon Chemie—Version 2.0, Stuttgart/New York: Georg Thieme Verlag 1999; T. W. Greene, P. G. Wuts, Protective Groups in Organic Synthesis, 3^{rd} ed., John Wiley, New York, 1999).

Preference is given in this connection to oxycarbonyl derivatives such as carbamates and especially the following groups: benzyloxycarbonyl, 4-bromobenzyloxycarbonyl, 2-chlorobenzyloxycarbonyl, 3-chlorobenzyloxycarbonyl, dichlorobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 4-nitrobenzyloxycarbonyl, 2-nitrobenzyloxycarbonyl, 2-nitro-4,5-dimethoxybenzyloxycarbonyl, 3,4,5-trimethoxybenzyloxycarbonyl, methoxycarbonyl, ethoxycarbonyl, propoxycarbonyl, isopropoxycarbonyl, butoxycarbonyl, isobutoxycarbonyl, tert-butoxycarbonyl, pentoxycarbonyl, isopentoxycarbonyl, hexoxycarbonyl, cyclohexoxycarbonyl, octoxycarbonyl, 2-ethylhexoxycarbonyl, 2-iodohexoxycarbonyl, 2-bromoethoxycarbonyl, 2-chloroethoxycarbonyl, 2,2,2-trichloroethoxycarbonyl, 2,2,2-trichloro-tert-butoxycarbonyl, benzhydryloxycarbonyl, bis(4-methoxyphenyl)methoxycarbonyl, phenacyloxycarbonyl, 2-trimethylsilylethoxycarbonyl, phenacyloxycarbonyl, 2-trimethylsilylethoxycarbonyl, 2-(di-n-butylmethylsilyl)ethoxycarbonyl, 2-triphenylsilylethoxycarbonyl, 2-(dimethyl-tert-butylsilyl)ethoxycarbonyl, methyloxycarbonyl, vinyloxycarbonyl, allyloxycarbonyl, phenoxycarbonyl, tolyloxycarbonyl, 2,4-dinitrophenoxycarbonyl, 4-nitrophenoxycarbonyl, 2,4,5-trichlorophenoxycarbonyl, naphthyloxycarbonyl, fluorenyl-9-methoxycarbonyl, valeroyl, isovaleroyl, butyryl, ethylthiocarbonyl, methylthiocarbonyl, butylthiocarboyl, tert-butylthiocarbonyl, phenylthiocarbonyl, benzylthiocarbonyl, methylaminocarbonyl, ethylaminocarbonyl, propylaminocarbonyl, isopropylaminocarbonyl, formyl, acetyl, propionyl, pivaloyl, 2-chloroacetyl, 2-bromoacetyl, 2-iodoacetyl, 2,2,2-trifluoroacetyl, 2,2,2-trichloroacetyl, benzoyl, 4-chlorobenzoyl, 4-methoxybenzoyl, 4-nitrobenzyl, 4-nitrobenzoyl, naphthylcarbonyl, phenoxyacetyl, adamantylcarbonyl, dicyclohexylphosphoryl, diphenylphosphoryl, dibenzylphosphoryl, di(4-nitrobenzyl)phosphoryl, phenoxyphenylphosphoryl, diethylphosphinyl, diphenylphosphinyl, phthaloyl, phthalimido or benzyloxymethylene.

Particular preference is given to tert-butyloxycarbonyl (Boc), 9-fluorenylmethyloxycarbonyl (FMOC), benzyloxycarbonyl (Cbz-/Z-) and allyloxycarbonyl (Aloc).

A symbol * on a bond denotes a chiral center.

Preference is given for the purposes of the present invention to compounds which correspond to the formula 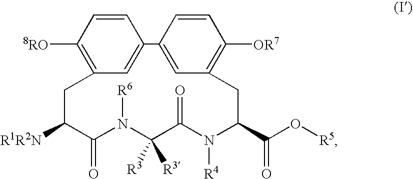
in which R¹ to R⁸ have the same meaning as in formula (I),
and the salts thereof, the solvates thereof and the solvates of the salts thereof.

Preference is given for the purposes of the present invention to compounds of the invention in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
   where R¹ apart from hydrogen may be substituted by 0, 1, 2 or 3 substituents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy and carboxyl,
R² is hydrogen or alkyl,
   where alkyl may be substituted by 0, 1, 2 or 3 substituents R²⁻¹, where the substituents R²⁻¹ are selected independently of one another from the group consisting of halogen, amino, alkylamino and dialkylamino, or
R¹ and R² together with the nitrogen atom to which they are bonded form a heterocycle which may be substituted by 0, 1 or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
   and in which one or more free amino groups in the side group of the amino acid may be substituted by alkyl, alkenyl, cycloalkyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen or C₁-C₆-alkyl,
R⁴ is hydrogen, C₁-C₆-alkyl or C₃-C₈-cycloalkyl,
R⁵ is alkyl, cycloalkyl, aryl, heteroaryl, heterocyclyl or a hydroxyl function-linked amino acid residue, where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
R⁶ is hydrogen, C₁-C₆-alkyl or C₃-C₈-cycloalkyl,
R⁷ is hydrogen or C₁-C₆-alkyl, and
R⁸ is hydrogen or C₁-C₆-alkyl.

Preference is given for the purposes of the present invention also to compounds of the invention in which
R¹ is hydrogen, alkyl, alkylcarbonyl, arylcarbonyl, heterocyclylcarbonyl, heteroarylcarbonyl, alkoxycarbonyl or a carbonyl-linked amino acid residue,
   where R¹ may be substituted by 0, 1 or 2 substituents R¹⁻¹, where the substituents R¹⁻¹ are selected independently of one another from the group consisting of halogen, trifluoromethyl, amino, alkylamino, dialkylamino, phenyl, 5- to 6-membered heteroaryl, 5- to 6-membered heterocyclyl, hydroxy and alkoxy,
R² is hydrogen or methyl,
R³ is aminocarbonylmethyl, 3-aminopropyl, 2-hydroxy-3-aminopropyl, 3-guanidinopropyl, 2-aminocarbonylethyl, 2-hydroxycarbonylethyl, 4-aminobutyl, hydroxymethyl, 2-hydroxyethyl or 4-amino-3-hydroxybutan-1-yl,
   and in which free amino groups in the side group of the amino acid may be substituted by alkyl, alkenyl, C₃-C₆-cycloalkyl, alkylcarbonyl, phenylcarbonyl, 5- to 6-membered heteroarylcarbonyl, 5- to 6-membered heterocyclylcarbonyl, alkoxycarbonyl, aminocarbonyl, alkylaminocarbonyl, dialkylaminocarbonyl, phenylaminocarbonyl, alkylsulfonyl, arylsulfonyl, 5- to 6-membered heterocyclylsulfonyl or 5- to 6-membered heteroarylsulfonyl,
R^{3′} is hydrogen,
R⁴ is hydrogen or methyl,
R⁵ is alkyl, C₃-C₆-cycloalkyl, phenyl, 5- to 6-membered heteroaryl, 5- to 6-membered heterocyclyl or a hydroxy function-linked amino acid residue,
   where in the case where R⁵ is alkyl, C₃-C₆-cycloalkyl or 5- to 6-membered heterocyclyl, the latter may be substituted by 0, 1 or 2 substituents R⁵⁻², where the substituents R⁵⁻² are selected independently of one another from the group consisting of alkyl, trifluoromethyl, amino, alkylamino, dialkylamino, C₃-C₆-cycloalkyl, phenyl, 5- to 6-membered heteroaryl, 5- to 6-membered heterocyclyl, hydroxy, alkoxy, carboxyl, alkoxycarbonyl, aminocarbonyl, alkylaminocarbonyl and dialkylaminocarbonyl, and
   where in the case where R⁵ is phenyl or 5- to 6-membered heteroaryl, the latter may be substituted by 0, 1 or 2 substituents R⁵⁻³, where the substituents R⁵⁻³ are selected independently of one another from the group consisting of halogen, trifluoromethyl, amino, alkylamino, dialkylamino, C₃-C₆-cycloalkyl, 5- to 6-membered heteroaryl, 5- to 6-membered heterocyclyl, hydroxy, alkoxy, carboxyl, alkoxycarbonyl, aminocarbonyl, alkylaminocarbonyl and dialkylaminocarbonyl,
R⁶ is hydrogen or methyl
R⁷ is hydrogen, and
R⁸ is hydrogen.

Preference is given for the purposes of the present invention also to compounds of the invention in which
R¹ is hydrogen, alkyl or alkylcarbonyl,
R² is hydrogen,
R³ is alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino, and in which one or more free amino groups in the side group of the amino acid may be substituted by alkyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is alkyl, cycloalkyl, aryl, heteroaryl or heterocyclyl, where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
   in which alkylamino and dialkylamino may be substituted by 0, 1 or 2 substituents R⁵⁻², where the substituents R⁵⁻² are selected independently of one another from the group consisting of hydroxy, amino, alkoxy, alkylamino and dialkylamino,
R⁶ is hydrogen,
R⁷ is hydrogen, C₁-C₆-alkyl, alkylcarbonyl or C₃-C₈-cycloalkyl, and
R⁸ is hydrogen.

Preference is given for the purposes of the present invention also to compounds of the invention in which
R¹ is hydrogen,
R¹ is hydrogen,
R³ is alkyl or the side group of an amino acid, in which alkyl may be substituted by 0, 1, 2 or 3 substituents R³⁻¹, where the substituents R³⁻¹ are selected independently of one another from the group consisting of amino, alkylamino, dialkylamino, cycloalkyl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, heteroaryl and heterocyclyl may be substituted by 0, 1 or 2 substituents R³⁻², where the substituents R³⁻² are selected independently of one another from the group consisting of alkyl and amino,
R^{3′} is hydrogen,
R⁴ is hydrogen, C₁-C₆-alkyl or C₃-C₈-cycloalkyl,
R⁵ is alkyl, cycloalkyl, aryl, heteroaryl or heterocyclyl, where R⁵ may be substituted by 0, 1, 2 or 3 substituents R⁵⁻¹, where the substituents R⁵⁻¹ are selected independently of one another from the group consisting of alkyl, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
   in which alkylamino and dialkylamino may be substituted by 0, 1 or 2 substituents R⁵⁻², where the substituents R⁵⁻² are selected independently of one another from the group consisting of hydroxy, amino, alkoxy, alkylamino and dialkylamino,
R⁶ is hydrogen,
R⁷ is hydrogen, and
R⁸ is hydrogen.

Preference is given for the purposes of the present invention also to compounds of the invention in which
R¹ is hydrogen,
R² is hydrogen,
R³ is aminocarbonylmethyl, 3-aminoprop-1-yl, 2-hydroxy-3-aminoprop-1-yl, 1-hydroxy-3-aminoprop-1-yl, 3-guanidinoprop-1-yl, 2-aminocarbonylethyl, 2-hydroxycarbonylethyl, 4-aminobut-1-yl, hydroxymethyl, 2-hydroxyethyl, 2-aminoethyl, 4-amino-3-hydroxybut-1-yl or (1-piperidin-3-yl)methyl,
R^{3′} is hydrogen,
R⁴ is hydrogen, methyl, ethyl, isopropyl or cyclopropyl,
R⁵ is alkyl or C₃-C₆-cycloalkyl, where R⁵ may be substituted by 0, 1, 2 or 3 substituents R⁵⁻¹, where the substituents R⁵⁻¹ are selected independently of one another from the group consisting of alkyl, amino, alkylamino, dialkylamino, cycloalkyl, hydroxy, alkoxy, carboxyl, alkoxycarbonyl, aminocarbonyl, alkylaminocarbonyl and dialkylaminocarbonyl,
   in which alkylamino and dialkylamino may be substituted by 0, 1 or 2 substituents R⁵⁻², where the substituents R⁵⁻² are selected independently of one another from the group consisting of hydroxy and amino,
R⁶ is hydrogen,
R⁷ is hydrogen, and
R⁸ is hydrogen.

Particular preference is given for the purposes of the present invention to compounds of the invention in which
R¹ is hydrogen,
R² is hydrogen,
R³ is 3-aminoprop-1-yl or 2-hydroxy-3-aminoprop-1-yl,
R^{3′} is hydrogen,
R⁴ is hydrogen or methyl,
R⁵ is C₁-C₄-alkyl, where alkyl may be substituted by 0, 1 or 2 substituents selected independently of one another from the group consisting of amino, hydroxy and carboxyl,
R⁶ is hydrogen,
R⁷ is hydrogen, and
R⁸ is hydrogen.

Preference is given for the purposes of the present invention also to compounds of the invention in which R¹ is hydrogen.

Preference is given for the purposes of the present invention also to compounds of the invention in which R² is hydrogen.

Preference is given for the purposes of the present invention also to compounds of the invention in which R³ is 3-aminoprop-1-yl or 2-hydroxy-3-aminoprop-1-yl.

Preference is given for the purposes of the present invention also to compounds of the invention in which R^{3′} is hydrogen.

Preference is given for the purposes of the present invention also to compounds of the invention in which R⁴ is hydrogen or methyl.

Preference is given for the purposes of the present invention also to compounds of the invention in which
R⁵ is alkyl or C₃-C₆-cycloalkyl where R⁵ may be substituted by 0, 1, 2 or 3 substituents R⁵⁻¹, where the substituents R⁵⁻¹ are selected independently of one another from the group consisting of alkyl, amino, alkylamino, dialkylamino, cycloalkyl, hydroxy, alkoxy, carboxyl, alkoxycarbonyl, aminocarbonyl, alkylaminocarbonyl and dialkylaminocarbonyl,
   in which alkylamino and dialkylamino may be substituted by 0, 1 or 2 substituents R⁵⁻², where the substituents R⁵⁻² are selected independently of one another from the group consisting of hydroxy and amino.

Preference is given for the purposes of the present invention also to compounds of the invention in which R⁵ is C₁-C₄-alkyl, where alkyl may be substituted by 0, 1 or 2 substituents independently of one another selected from the group consisting of amino, hydroxy and carboxyl.

Preference is given for the purposes of the present invention also to compounds of the invention in which R⁶ is hydrogen.

Preference is given for the purposes of the present invention also to compounds of the invention in which R⁷ is hydrogen.

Preference is given for the purposes of the present invention also to compounds of the invention in which R⁸ is hydrogen.

The invention further relates to a process for preparing the compounds of the formula (I) or their salts, where compounds of the formula 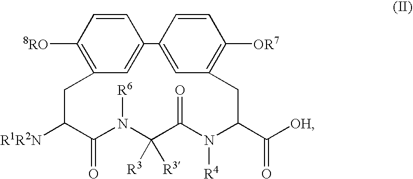
in which R¹ to R⁴ and R⁶ to R⁸ have the meaning indicated above, where the compounds of the formula (II) may where appropriate be in activated form (as acyl donor),
are reacted with compounds of the formula
HO—R⁵ (III),
in which
R⁵ has the meaning indicated above.

Where appropriate, reaction of compounds of the formula (II) with compounds of the formula (III) is preceded by blocking of reactive functionalities (e.g. free amino functions or hydroxy functions) in compounds of the formula (II) by protective groups. This takes place by standard methods of protective group chemistry. Preference is given to acid-labile protective groups on R¹ (or R²), or as substituents in the radicals R³ and R^{3′}, with particular preference for Boc. Reactive functionalities in R⁵ of compounds of the formula (III) are introduced already protected into the synthesis. Preference is given to acid-labile protective groups (e.g. Boc) or protective groups which can be eliminated by hydrogenolysis (e.g. benzyl or benzyloxycarbonyl). After reaction has taken place to give compounds of the formula (I), the protective groups can be eliminated by deprotection reactions. This takes place by standard methods of protective group chemistry. Deprotection reactions under acidic conditions are preferred.

If, for example, R² in compounds of the formula (I) is a protective group which can be selectively eliminated, deprotection (e.g. hydrogenolysis in the case of R² equal Z) can be followed by functionalization of the exposed amino function (R² equal hydrogen) with the desired substituent R².

Suitable for converting the carboxylic acid function in formula (II) in the activated form are, for example, carbodiimides such as, for example, N,N′-diethyl-, N,N′-dipropyl-, N,N′-diisopropyl-(DIC) and N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole. The activation takes place where appropriate in the presence of 4-dimethylaminopyridine.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane, dimethylformamide and acetonitrile are particularly preferred.

Reactions with activation by EDC or DIC in absolute acetonitrile, dimethylformamide or dichlormethane at low temperature (−10° C.) in the presence of 4-dimethylaminopyridine are preferred.

The invention further relates to an alternative process for preparing the compounds of the formula (I) or their salts, characterized in that compounds of the formula (II) can also be reacted with compounds of the formula (III) with acid catalysis. For this purpose, the compounds of the formula (II) are mixed with an excess of anhydrous alcohol HO—R⁵, where appropriate in the presence of an inert solvent, and at room temperature or up to the boiling point of the solution an acid (preferably a mineral acid) or acid-liberating reagents (e.g. thionyl chloride) are added and reacted to give compounds of the formula (I).

Solvents suitable in this case are inert organic solvents which are not changed under the reaction conditions. These include halohydrocarbons such as dichloromethane or trichloromethane, hydrocarbons such as benzene, toluene, tetrahydrofuran, dioxane. It is likewise possible to employ mixtures of the solvents.

The compounds of the formula (III) are known or can be prepared in analogy to known processes.

The compounds of the formula (II) are known or can be prepared by hydrolyzing the ester function in compounds of the formula 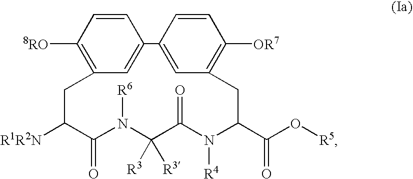
in which
R¹ to R⁴ and R⁶ to R⁸ have the meaning indicated above, and
R⁵ is benzyl, alkyl or allyl.

The ester cleavage takes place where R⁵ is benzyl preferably with hydrogen in the presence of palladium on carbon.

Suitable solvents in this case are organic solvents which are not changed under the reaction conditions. These include halohydrocarbons such as dichloromethane or trichloromethane, hydrocarbons such as tetrahydrofuran, dioxane, dimethylformamide, acetic acid, mixtures of acetic acid and water, or alcohols (with preference for methanol, ethanol and isopropanol), where appropriate in the presence of one or more acid equivalents. It is likewise possible to employ mixtures of the solvents. Mixtures of acetic acid, water and ethanol or THF are particularly preferred.

The ester cleavage takes place when R⁵ is allyl preferably in the presence of palladium(0) catalysts by standard methods of protective group chemistry.

Suitable solvents are degassed (oxygen-purged) organic solvents which are not changed under the reaction conditions. These include halohydrocarbons such as dichloromethane or trichloromethane, hydrocarbons such as tetrahydrofuran, dioxane and dimethylformamide, where appropriate in the presence of one or more acid equivalents.

An alternative possibility is for the esters (R⁵ equal to benzyl, alkyl) also to be cleaved by basic hydrolysis to give the corresponding carboxylic acids.

Aqueous lithium or sodium hydroxide are preferably employed as bases.

Suitable solvents in this case are organic solvents which are partly or infinitely miscible with water. These include alcohols (with preference for methanol and ethanol), tetrahydrofuran, dioxane and dimethylformamide. It is likewise possible to employ mixtures of these solvents. Methanol, tetrahydrofuran and dimethylformamide are particularly preferred.

The invention further relates to an alternative process for preparing the compounds of the formulae (I) and (Ia) or their salts, characterized in that compounds of the formula 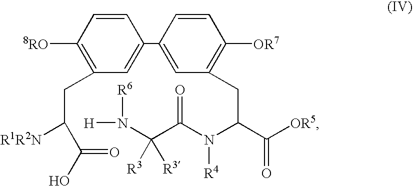
in which
R¹ to R⁸ have the meaning indicated above,
   where these are where appropriate in activated form, are cyclized under peptide-coupling conditions.

An alternative possibility is a multistage process in which compounds of the formula 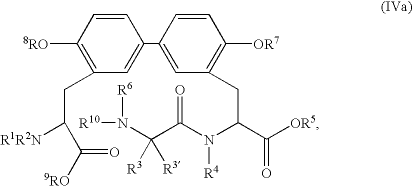
in which
R¹ to R⁸ have the meaning indicated above,
R⁹ after activation is pentafluorophenol, and
R¹⁰ is an amine protective group (preferably Boc),
   are converted by protective group elimination of the amine protective group (to give R¹⁰ equal to hydrogen) and subsequent cyclization under basic conditions into compounds of the formula (I) and (Ia).

Suitable for converting the compounds into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC) (where appropriate in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate or O-(benzotriazol-1-yl)-N,N,N′,N′-tetramethyluronium hexafluorophosphate (HBTU), or 2-(2-oxo-1-(2H)-pyridyl)-1,1,3,3-tetramethyluronium tetrafluoroborate (TPTU), or O-(7-azabenzotriazol-1-yl)-N,N,N′,N′-tetramethyluronium hexafluorophosphate (HATU), or benzotriazol-1-yloxytris(dimethylamino)phosphonium hexafluorophosphate (BOP), or mixtures of these with bases, where appropriate in the presence of 1-hydroxybenzotriazole (HOBt).

Examples of bases are alkali metal carbonates, such as, for example, sodium or potassium carbonate, or bicarbonate, or preferably organic bases such as trialkylamines, e.g. triethylamine, N-methylmorpholine, N-methylpiperidine, 4-dimethylaminopyridine or diisopropylethylamine.

Solvents which are suitable in this case are inert organic solvents which are not changed under the reaction conditions. These include halohydrocarbons such as dichloromethane or trichloromethane, hydrocarbons such as benzene, toluene, tetrahydrofuran, dioxane, dimethylformamide or acetonitrile. It is likewise possible to employ mixtures of the solvents. Dichloromethane and dimethylformamide are particularly preferred.

Preparation of the compounds of the invention of the formula (I) can take place as shown in the following synthesis scheme.

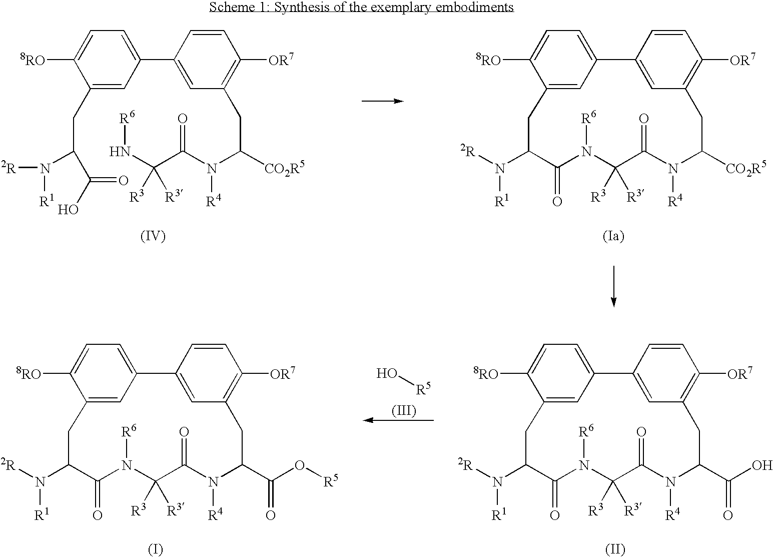

The compounds of the formula (IV) are known, can be prepared in analogy to known processes or by reacting compounds of the formula 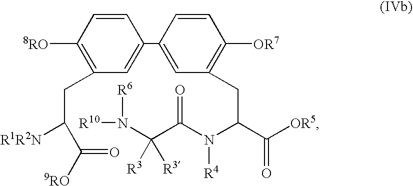
in which
R¹ to R⁸ and R¹⁰ have the meaning indicated above, and
R⁹ is a silyl protective group, in particular 2-(trimethylsilyl)ethyl,
   after elimination of the protective group on R¹⁰, with fluoride, in particular with tetrabutylammonium fluoride.

Solvents suitable in this case are inert organic solvents which are not changed under the reaction conditions. These include halohydrocarbons such as dichloromethane, hydrocarbons such as benzene, toluene, tetrahydrofuran, dioxane and dimethylformamide. It is likewise possible to employ mixtures of the solvents. Preferred solvents are tetrahydrofuran and dimethylformamide.

The compounds of the formula (IVb) are known, can be prepared in analogy to known processes or by reacting compounds of the formula 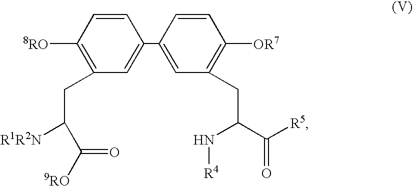
in which
R¹, R², R⁴, R⁵, R⁷ and R⁸ have the meaning indicated above, and
R⁹ is a silyl protective group, in particular 2-(trimethylsilyl)ethyl,
   with compounds of the formula (VI) 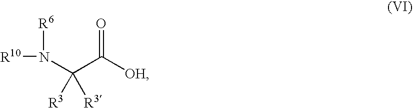
   in which
R³, R^{3′}, R⁶ and R¹⁰ have the meaning indicated above,
   where the compounds may where appropriate be in activated form.

Suitable for converting the compounds into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC) (where appropriate in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or 0-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or benzotriazol-1-yloxytris(dimethylamino)phosphonium hexafluorophosphate (BOP), or mixtures of these with bases, where appropriate with the addition of coupling additives such as 1-hydroxybenzotriazole (HOBt).

Examples of bases are alkali metal carbonates, such as, for example, sodium or potassium carbonate, or bicarbonate, or preferably organic bases such as trialkylamines, e.g. triethylamine, N-methylmorpholine, N-methylpiperidine, 4-dimethylaminopyridine or diisopropylethylamine.

Solvents which are suitable in this case are inert organic solvents which are not changed under the reaction conditions. These include halohydrocarbons such as dichloromethane or trichloromethane, hydrocarbons such as benzene, toluene, tetrahydrofuran, dioxane or dimethylformamide. It is likewise possible to employ mixtures of the solvents. Anhydrous dichloromethane and dimethylformamide are particularly preferred.

Reaction in the presence of a HATU and N,N-diisopropylethylamine is particularly preferred.

The compounds of the formula (VI) are known or can be prepared in analogy to known processes.

The compounds of the formula (V) and their salts (e.g. hydrochlorides) are known, can be prepared in analogy to known processes or by deprotection on R¹¹ of compounds of the formula 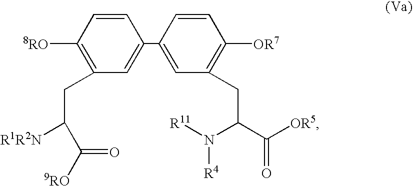
in which
R¹, R², R⁴, R⁵, R⁷ and R⁸ have the meaning indicated above,
R⁹ is a silyl protective group, and
R¹¹ is an amino protective group, in particular Boc.

This takes place by standard methods of protective group chemistry, preferably with hydrogen chloride in dioxane when R¹¹ is Boc.

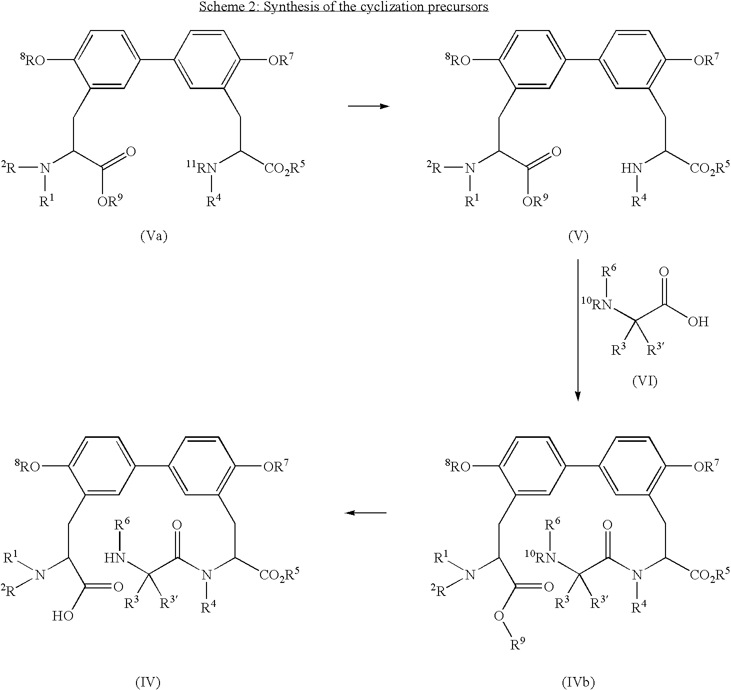

The compounds of the formula (Va) are known, can be prepared in analogy to known processes or by reacting compounds of the formula 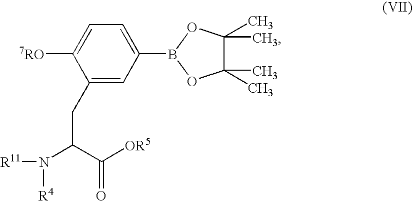
in which
R⁴, R⁵ and R⁷ have the meaning indicated above, and
R¹¹ is an amino protective group (preferably Boc),
   with compounds of the formula 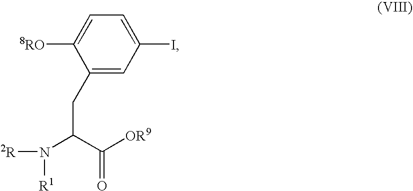
   in which
R¹, R² and R⁸ have the meaning indicated above, and
R⁹ is a silyl protective group, in particular 2-(trimethylsilyl)ethyl.

The reaction, known as the Suzuki reaction (Synlett 1992, 207-210; Chem. Rev. 1995, 95, 2457-2483), takes place in the presence of palladium catalysts, and a base, preferably in the presence of bis(diphenylphosphino)ferrocene-palladium(II) chloride and cesium carbonate.

Suitable solvents in this case are inert organic solvents which are not changed under the reaction conditions. These include hydrocarbons such as benzene, toluene, tetrahydrofuran, dioxane, dimethylformamide and dimethyl sulfoxide. It is likewise possible to employ mixtures of the solvents. Dimethylformamide and dimethyl sulfoxide are particularly preferred.

The compounds of the formula (VII) are known, can be prepared in analogy to known processes, or by reacting compounds of the formula 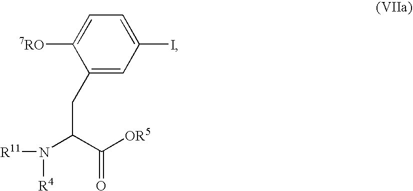
in which
R⁴, R⁵ and R⁷ have the meaning indicated above, and
R¹¹ is an amino protective group (preferably Boc),
with bis(pinacolato)diboron. This reaction, known as a special variant of the Suzuki reaction (J. Org. Chem. 1995, 7508-7510; Tetrahedron Lett., 1997, 3841-3844), takes place in the presence of palladium catalysts and a base, preferably in the presence of bis(diphenylphosphino)ferrocenepalladium(II) chloride and of potassium acetate.

Suitable solvents in this case are inert organic solvents which are not changed under the reaction conditions. These include hydrocarbons such as benzene, toluene, tetrahydrofuran, dioxane, dimethylformamide and dimethyl sulfoxide. It is likewise possible to employ mixtures of the solvents. Dimethylformamide and dimethyl sulfoxide are particularly preferred.

The compounds of the formula (VIIa) are known, can be prepared in analogy to known processes, or by reacting compounds of the formula 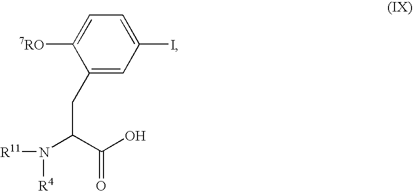
in which
R⁴ and R⁷ have the meaning indicated above, and
R¹¹ is an amino protective group (preferably Boc),
   after activation of the free carboxylate function with R⁵—OH alcohols preferably in the presence of 4-dimethylaminopyridine.

Suitable for converting the carboxylic acids into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane and acetonitrile are particularly preferred.

Reactions with activation by EDC or DIC in absolute acetonitrile or dichloromethane at low temperature (−10° C.) in the presence of 4-dimethylaminopyridine are preferred.

The compounds of the formula (VIII) are known, can be prepared in analogy to known processes, or by reacting compounds of the formula 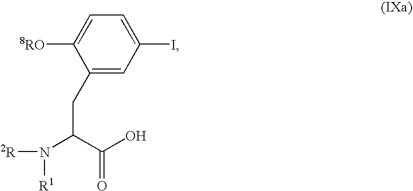
in which
R¹, R² and R⁸ have the meaning indicated above,
   after activation of the free carboxylate function with R⁹—OH (preferably 2-trimethylsilylethanol) in the presence of 4-dimethylaminopyridine.

Suitable for converting the carboxylic acids into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane and acetonitrile are particularly preferred.

Reactions with activation by EDC or DIC in absolute acetonitrile or dichloromethane at low temperature (−10° C.) in the presence of 4-dimethylaminopyridine are preferred.

The carboxylic acids of the formula (IXa) are known, can be prepared in analogy to known processes, or by deprotecting compounds of the formula 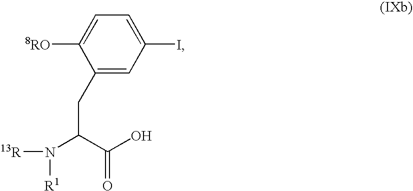
in which
R¹ and R⁸ have the meaning indicated above, and
R¹³ is an amino protective group, in particular Boc,
   in the first stage on R³. This takes place by standard methods of protective group chemistry, when R¹³ is Boc preferably with anhydrous hydrogen chloride in dioxane or with trifluoroacetic acid in dichloromethane in the presence of small amounts of water. The resulting free amine 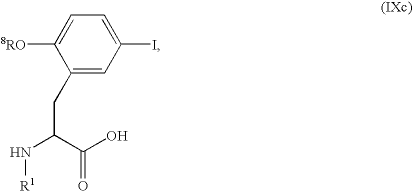
   in which
R¹ and R⁸ have the meaning indicated above,
   where the amine may where appropriate be in the form of a salt, preferably hydrochloride or trifluoroacetate,
   is reacted in the second stage with R²—X, in which R² has the meaning indicated above, and X is a leaving group, in the presence of a base in inert solvents, where appropriate in the presence of potassium iodide, preferably in a temperature range from 0° C. via room temperature to reflux of the solvent under atmospheric pressure. Mesylate, tosylate, succinate or halogen are preferred for X, with chlorine, bromine or iodine being preferred for halogen.

Examples of bases are alkali metal carbonates such as, for example, sodium or potassium carbonate, or bicarbonate, or organic bases such as trialkylamines, e.g. triethylamine, N-methylpiperidine, 4-dimethylaminopyridine or diisopropylethylamine.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane, acetone or dimethylformamide. It is likewise possible to use mixtures of the solvents. Dimethylformamide and dichloromethane are particularly preferred.

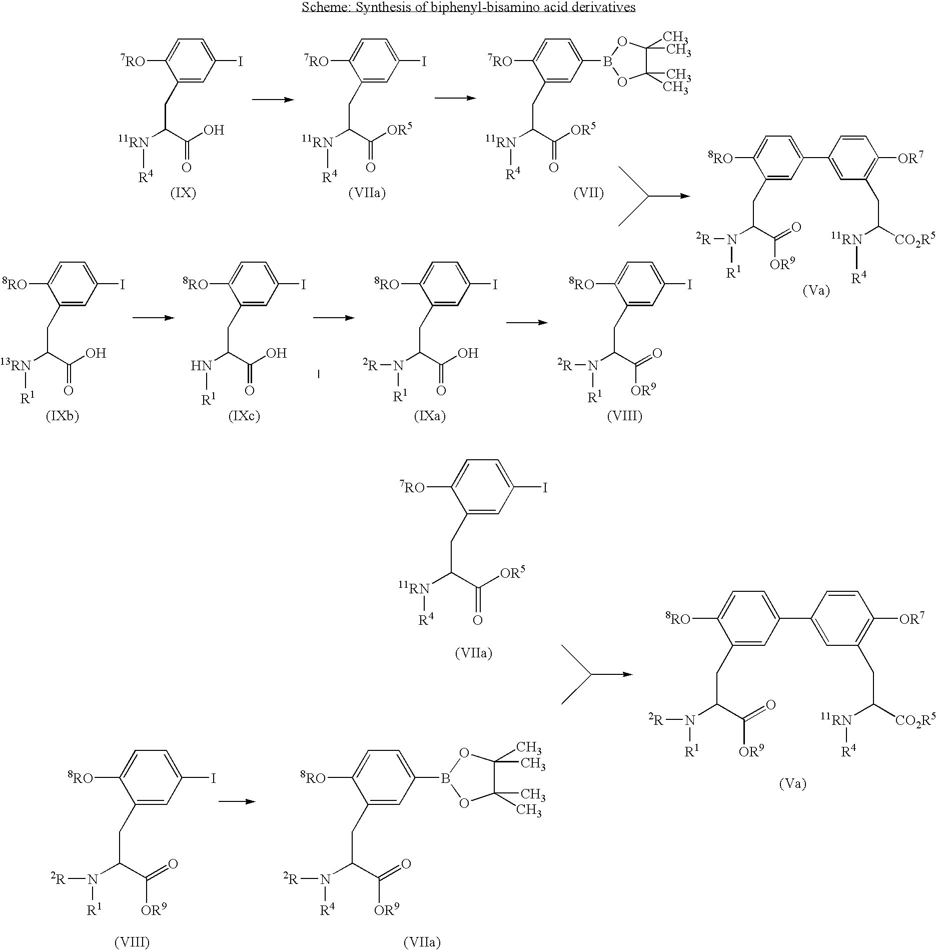

R² can optionally be a protective group (e.g. Z, i.e. benzyloxycarbonyl or Aloc, i.e. allyloxycarbonyl).

In an alternative process, the compounds of the formula (Va) can be prepared by reacting compounds of the formula 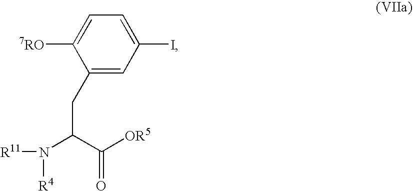
in which
R⁴, R⁵ and R⁷ have the meaning indicated above, and
R¹¹ is an amino protective group (preferably Boc),
   with compounds of the formula 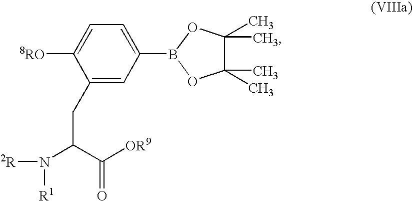
   in which
R¹, R² and R⁸ have the meaning indicated above, and
R⁹ is an silyl protective group, in particular 2-(trimethylsilyl)ethyl.

The reaction, known as the Suzuki reaction (Synlett 1992, 207-210; Chem. Rev. 1995, 95, 2457-2483), takes place in the presence of palladium catalysts and a base, preferably in the presence of bis(diphenylphosphino)ferrocenepalladium(II) chloride and cesium carbonate.

Suitable solvents in this case are inert organic solvents which are not changed under the reaction conditions. These include hydrocarbons such as benzene, toluene, tetrahydrofuran, dioxane, dimethylformamide and dimethyl sulfoxide. It is likewise possible to employ mixtures of the solvents. Dimethylformamide and dimethyl sulfoxide are particularly preferred.

The compounds of the formula (VIIIa) can be prepared from the compounds of the formula (VIII) by the process described for compounds (VII).

The enantiopure compounds of the formulae (IX) and (IXb) are known or can be obtained from racemic precursors by known processes, such as, for example, crystallization with chiral amine bases or by chromatography on chiral stationary phases.

The compounds of the formulae (IX) and (IXb) are known, can be prepared in analogy to known processes, or by decarboxylating compounds of the formulae 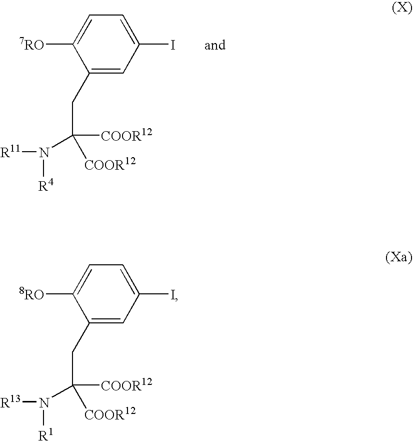
in which
R⁴ and R⁷ and R¹ and R⁸ have the meaning indicated above,
R¹¹ and R¹³ are an amino protective group, and
R¹² is alkyl particularly preferably ethyl).

This reaction preferably takes place in basic medium in a water-ethanol mixture.

The compounds of the formulae (X) and (Xa) are known, can be prepared in analogy to known processes, or by reacting compounds of the formulae 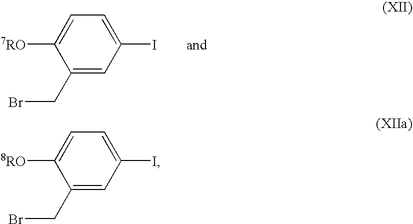
in which
R⁷ and R⁸ have the meaning indicated above,
   with compounds respectively of the formulae 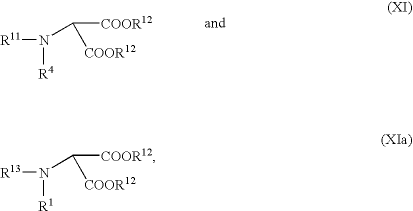
   in which
R⁴ and R¹ have the meaning indicated above,
R¹¹ and R¹³ are an amino protective group, and
R¹² is alkyl (particularly preferably ethyl).

This reaction preferably takes place with alkali metal alcoholate in lower aliphatic alcohol, in particular with sodium ethoxide in ethanol.

The compounds of the formulae (XII) and (XIIa) are known, can be prepared in analogy to known processes, or by reacting compounds of the formulae 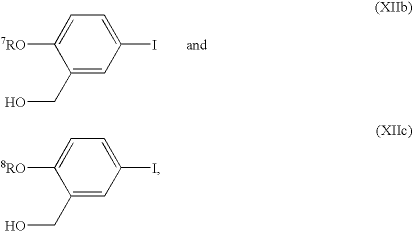
in which
R⁷ and R⁸ have the meaning indicated above,
   with phosphorus tribromide. The reaction preferably takes place in toluene.

The compounds of the formulae (XIIb) and (XIIc) are known, can be prepared in analogy to known processes, or by reducing compounds of the formulae 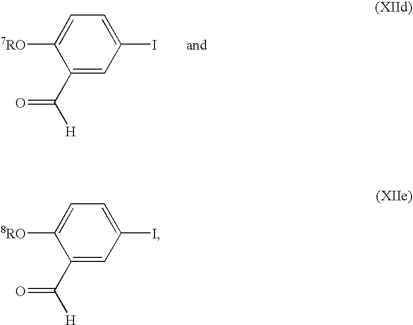
in which
R⁷ and R⁸ have the meaning indicated above.

The reduction preferably takes place with diisobutylaluminum hydride solution in dichloromethane with subsequent addition of a saturated potassium sodium tartrate solution.

The compounds of the formulae (XIId) and (XIIe) are known, can be prepared in analogy to known processes, or by reacting 2-hydroxy-5-iodobenzaldehyde with compounds respectively of the formulae
R⁷—X
and
R⁸—X
in which
R⁷ and R⁸ have the meaning indicated above, and
X is a leaving group, in inert solvents, where appropriate in the presence of a base, where appropriate in the presence of potassium iodide, preferably in a temperature range from room temperature to reflux of the solvent under atmospheric pressure. Mesylate, tosylate or halogen are preferred for X, with chlorine, bromine or iodine being preferred for halogen.

Examples of inert solvents are halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile, preferably tetrahydrofuran, methylene chloride, acetone, 2-butanone, acetonitrile, dimethylformamide or 1,2-dimethoxyethane. Dimethylformamide is preferred.

Examples of bases are alkali metal carbonates such as cesium carbonate, sodium or potassium carbonate, or sodium or potassium methanolate, or sodium or potassium ethanolate or potassium tert-butoxide, or amides such as sodamide, lithiumbis(trimethylsilyl)amide or lithiumdiispropylamide, or organometallic compounds such as butyllithium or phenyllithium, tertiary amine bases such as triethylamine or diisopropylethylamine, or other bases such as sodium hydride, DBU, preferably potassium tert-butoxide, cesium carbonate, DBU, sodium hydride, potassium carbonate or sodium carbonate. Potassium carbonate is preferred.

The compounds of the formulae (XIII) and (XIIIa) are known or can be prepared in analogy to known processes.

The preparation of the compounds of the invention can be illustrated by the following synthesis scheme. In this, to improve clarity, the roman numerals used in the description are retained but the scheme shows in some cases specific embodiments, in particular R¹² in (XI) and (XIa) is ethyl and R¹¹ and R¹³ are Boc.

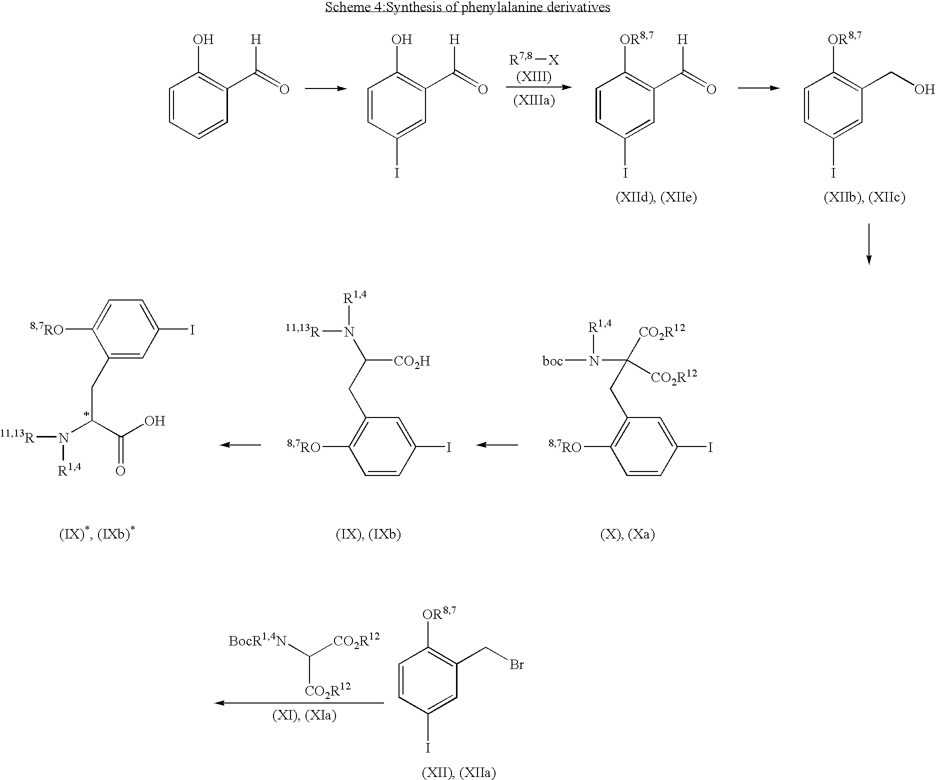

The compounds of the invention show a valuable range of pharmacological and pharmacokinetic effects which could not have been predicted. Preferably used for this purpose are compounds of the formula (I) which have a maximum inhibitory concentration (MIC) in relation to the appropriate bacteria of less than 100, in particular 50, very especially less than 10 μM. It is likewise preferred to use compounds of the formula (I) which have an IC₅₀ in the appropriate tests of less than 100, in particular 50, very especially less than 10 μM.

They are therefore suitable for use as medicaments for the treatment and/or prophylaxis of diseases in humans and animals.

The compounds of the invention can, because of their pharmacological properties, be employed alone or in combination with other active ingredients for the treatment and/or prevention of infectious diseases, in particular of bacterial infections.

It is possible for example to treat and/or prevent local and/or systemic diseases caused by the following pathogens or by mixtures of the following pathogens:

Gram-positive cocci, e.g. staphylococci (Staph. aureus, Staph. epidermidis) and streptococci (Strept. agalactiae, Strept. faecalis, Strept. pneumoniae, Strept. pyogenes); gram-negative cocci (neisseria gonorrhoeae) and gram-negative rods such as enterobacteriaceae, e.g. Escherichia coli, Hemophilus influenzae, Citrobacter (Citrob. freundii, Citrob. divemis), Salmonella and Shigella; also klebsiellas (Klebs. pneumoniae, Klebs. oxytocy), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. The antibacterial range also includes the genus Pseudomonas (Ps. aeruginosa, Ps. maltophilia) and strictly anaerobic bacteria such as, for example, Bacteroides fragilis, representatives of the genus Peptococcus, Peptostreptococcus, and the genus Clostridium; also mycoplasmas (M. pneumoniae, M. hominis, M. urealyticum) and mycobacteria, e.g. Mycobacterium tuberculosis.

The above list of pathogens is merely by way of example and is by no means to be interpreted restrictively. Examples which may be mentioned of diseases which may be caused by the pathogens or mixed infections and which may be prevented, improved or cured by the preparations of the invention which can be used topically are:

infectious diseases in humans, such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, bacterial infections can also be treated in other species. Examples which may be mentioned are:

pigs: coli diarrhea, enterotoxamia, sepsis, dysentery, salmonellosis, metritis-mastitis-agalactiae syndrome, mastitis;

ruminants (cattle, sheep, goats): diarrhea, sepsis, bronchopneumonia, salmonellosis, pasteurellosis, mycoplasmosis, genital infections;

horses: bronchopneumonias, joint ill, puerperal and postpuerperal infections, salmonellosis;

dogs and cats: bronchopneumonia, diarrhea, dermatitis, otitis, urinary tract infections, prostatitis;

poultry (chickens, turkeys, quail, pigeons, ornamental birds and others): mycoplasmosis, E. coli infections, chronic airway disorders, salmonellosis, pasteurellosis, psittacosis.

It is likewise possible to treat bacterial diseases in the rearing and management of productive and ornamental fish, in which case the antibacterial spectrum is extended beyond the pathogens mentioned above to further pathogens such as, for example, Pasteurella, Brucella, Campylobacter, Listeria, Erysipelothris, corynebacteria, Borellia, Treponema, Nocardia, Rikettsie, Yersinia.

The present invention additionally relates to compounds of the general formula (I) for controlling diseases, especially bacterial diseases, to medicaments comprising compounds of the formula (I) and excipients, and to the use of compounds of the formula (I) for producing a medicament for the treatment of bacterial diseases.

The present invention further relates to a method for controlling bacterial infections in humans and animals by administration of an antibacterially effective amount of at least one compound of the formula (I).

The present invention further relates to medicaments which comprise at least one compound of the invention, preferably together with one or more pharmacologically acceptable excipients or carriers, and to the use thereof for the aforementioned purposes.

The active ingredient may act systemically and/or locally. For this purpose, it can be administered in a suitable manner such as, for example, by the oral, parenteral, pulmonary, nasal, sublingual, lingual, buccal, rectal, transdermal, conjunctival or otic route or as implant.

The active ingredient can be administered in administration forms suitable for these administration routes.

Suitable for oral administration are known administration forms which deliver the active ingredient rapidly and/or in a modified manner, such as, for example, tablets (uncoated and coated tablets, e.g. tablets provided with coatings resistant to gastric juice, or film-coated tablets), capsules, sugar-coated tablets, granules, pellets, powders, emulsions, suspensions, solutions and aerosols.

Parenteral administration can take place with avoidance of an absorption step (intravenous, intraarterial, intracardiac, intraspinal or intralumbal) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates and sterile powders.

Suitable for the other administration routes are, for example, pharmaceutical forms for inhalation (inter alia powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active ingredients can be converted in a manner known per se into the stated administration forms. This takes place with use of inert nontoxic, pharmaceutically suitable excipients. These include inter alia carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colors (e.g. inorganic pigments such as iron oxides) or masking tastes and/or odors.

It has generally proved advantageous on parenteral administration to administer amounts of about 5 to 250 mg/kg of body weight every 24 h to achieve effective results. The amount on oral administration is about 5 to 100 mg/kg of body weight every 24 h.

It may nevertheless be necessary where appropriate to deviate from the stated amounts, in particular as a function of the body weight, administration route, individual behavior towards the active ingredient, nature of the preparation and time or interval over which administration takes place. Thus, it may be sufficient in some cases to make do with less than the aforementioned minimum amount, whereas in other cases the stated upper limit must be exceeded. Where larger amounts are administered, it may be advisable to divide these into a plurality of single doses over the day.

The percentage data in the following tests and examples are percentages by weight unless indicated otherwise; parts are parts by weight. Solvent ratios, dilution ratios and concentration data for liquid/liquid solutions are in each case based on volume.

### A. EXAMPLES

Abbreviations Used:

Aloc allyloxycarbonyl
aq. aqueous
Bn benzyl
Boc tert-butoxycarbonyl
CDCl₃ chloroform
CH cyclohexane
d doublet (in ¹H-NMR)
dd doublet of doublets
DCM dichloromethane
DCC dicyclohexylcarbodiimide
DIC diisopropylcarbodiimide
DIPEA diisopropylethylamine
DMSO dimethyl sulfoxide
DMAP 4-N,N-dimethylaminopyridine
DMF dimethylformamide
EA ethyl acetate (acetic acid ethyl ester)
EDC N′-(3-dimethylaminopropyl)-N-ethylcarbodiimide×HCl
ESI electrospray ionization (in MS)
HATU O-(7-azabenzotriazol-1-yl)-N,N,N′,N′-tetramethyluronium hexafluoro-phosphate
HBTU O-(benzotriazol-1yl)-N,N,N′,N′-tetramethyluronium hexafluorophosphate
HOBt 1-hydroxy-1H-benzotriazole×H₂O
h hour(s)
HPLC high pressure, high performance liquid chromatography
LC-MS coupled liquid chromatography-mass spectroscopy
m multiplet (in ¹H-NMR)
min minutes
MS mass spectroscopy
MeOH methanol
NMR nuclear magnetic resonance spectroscopy
MTBE methyl tert-butyl ether
Pd/C palladium/carbon
q quartet (in ¹H-NMR)
R_{f} retention index (in TLC)
RT room temperature
Rₜ retention time (in HPLC)
s singlet (in ¹H-NMR)
sat. saturated
t triplet (in ¹H-NMR)
TBS tert-butyldimethylsilyl
THF tetrahydrofuran
TMSE 2-(trimethylsilyl)ethyl
TPTU 2-(2-oxo-1(2H)pyridyl)-1,1,3,3-tetramethyluronium tetrafluoroborate
Z benzyloxycarbonyl
   General LC-MS and HPLC Methods

Method 1 (HPLC): column: Kromasil C18, L-R temperature: 30° C.; flow rate: 0.75 ml/min; eluent A: 0.01 M HClO₄, eluent B: acetonitrile, gradient:→0.5 min 98% A→4.5 min 10% A→6.5 min 10% A.

Method 2 (HPLC): column: Kromasil C18, 60*2 mm, L-R temperature: 30° C.; flow rate: 0.75 ml/min; eluent A: 0.01 M H₃PO₄, eluent B: acetonitrile, gradient:→0.5 min 90% A→4.5 min 10% A→6.5 min 10% A.

Method 3 (HPLC): column: Kromasil C18, 60*2 mm, L-R temperature: 30° C.; flow rate: 0.75 ml/min; eluent A: 0.005 M HClO₄, eluent B: acetonitrile, gradient:→0.5 min 98% A→4.5 min 10% A→6.5 min 10% A.

eluent B: acetonitrile+0.05% formic acid, gradient: 0.0 min 5% B→12 min→100% B→15 min 100% B.

Method 11 (LC-MS): MAT 900, Finnigan MAT, Bremen; column: X-terra 50 mm×2.1 mm, 2.5 μm; temperature: 25° C.; flow rate: 0.5 ml/min; eluent A: water+0.01% formic acid, eluent B: acetonitrile+0.01% formic acid, gradient: 0.0 min 10% B→15 min 90% B→30 min 90% B.

Method 12 (LC-MS): TSQ 7000, Finnigan MAT, Bremen; column: Inertsil ODS3 50 mm×2.1 mm, 3 μm; temperature: 25° C.; flow rate: 0.5 ml/min; eluent A: water+0.05% formic acid, eluent B: acetonitrile+0.05% formic acid, gradient: 0.0 min 15% B→15 min→100% B→30 min 100% B.

Method 13 (LC-MS): 7 Tesla Apex II with external electrospray ion source, Bruker Daltronics; column: X-terra C18 50 mm×2.1 mm, 2.5 μm; temperature: 25° C.; flow rate: 0.5 ml/min; eluent A: water+0.1% formic acid, eluent B: acetonitrile+0.1% formic acid, gradient: 0.0 min 5% B→13 min→100% B→15 min 100% B.

Method 14 (HPLC): column: X-Terra™ from Waters, RP₈, 5 μm, 3.9×150 mm; start: 95% A, 5% B; 12 min: 5% A, 95% B. Eluent A: water+0.01% trifluoroacetic acid; eluent B: acetonitrile+0.01% trifluoroacetic acid; flow rate: 1.2 ml/min.

Method 15 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 16 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→2.0 min 95% B→4.0 min 95% B; oven: 35° C.; flow rate: 0.0 min 1.0 ml/min→2.0 min 3.0 ml/min→4.0 min 3.0 ml/min; UV detection: 210 nm.

Method 17 (LC-MS): Instrument: Micromass Platform LCZ with HPLC Agilent series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 210 nm.

Method 18 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 19 (LC-MS): MS instrument type: Micromass ZQ; HPLC instrument type: Waters Alliance 2790; column: Uptisphere C 18, 50 mm×2 mm, 3.0 μM; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 20 (LC-MS): MS instrument type: Micromass ZQ; HPLC instrument type: HP1100 series; UV DAD column: Grom-Sil 120 ODS-4 HE, 50 mm×2.0 mm, 3.0 μm; eluent A: water+500 μl of 50% formic acid/l, eluent B: acetonitrile+500 μl of 50% formic acid/l; gradient: 0.0 min 0% B→2.9 min 70% B→3.1 min 90% B→4.5 min 90% B; oven: 50° C.; flow rate: 0.8 ml/min; UV detection: 210 nm.

Method 21 (LC-MS): MS instrument type: Micromass ZQ; HPLC instrument type: Waters Alliance 2795; column: Phenomenex Synergi 2μ Hydro-RP Mercury 20×4 mm; eluent A: 1 l of water+0.5 ml of 50% formic acid, eluent B: 1 l of acetonitrile+0.5 ml of 50% formic acid; gradient: 0.0 min 90% A (flow rate: 1 ml/min)→2.5 min 30% A (flow rate: 2 ml/min)→3.0 min 5% A (flow rate: 2 ml/min)→4.5 min 5% A (flow rate: 2 ml/min); oven: 50° C.; UV detection: 210 nm.

Method 22 (LC-MS): MS instrument type: Micromass ZQ; HPLC instrument type: HP 1100 Series; UV DAD; column: Grom-Sil 120 ODS4 HE 50×2 mm, 3.0 μm; eluent A: water+500 μl of 50% formic acid/l, eluent B: acetonitrile+500 μl of 50% formic acid/l; gradient: 0.0 min 70% B→4.5 min 90% B; oven: 50° C.; flow rate: 0.8 ml/min, UV detection: 210 nm.

Method 23 (LC-MS): Instrument: Micromass Quattro LCZ with HPLC Agilent Series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 24 (LC-MS): MS apparatus type: Micromass ZQ; HPLC apparatus type: Waters Alliance 2790; column: Grom-Sil 120 ODS4 HE 50×2 mm, 3.0 μm; eluent A: water+500 μl of 50% formic acid; eluent B: acetonitrile+500 μl of 50% formic acid/l; gradient: 0.0 min 5% B→2.0 min 40% B→4.5 min 90% B→5.5 min 90% B; oven: 45° C.; flow rate: 0.0 min 0.75 ml/min→4.5 min 0.75 ml 5.5 min→5.5 min 1.25 ml; UV detection: 210 nm.

Method 25 (HPLC): Instrument: HP 1100 with DAD detection; column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 15 min 90% B; flow rate: 0.75 ml/min; temp.: 30° C.; UV detection: 210 nm.

Chemical Synthesis of the Examples

Synthesis of the Starting Compounds:

### Synthesis of substituted phenylalanine derivatives with (−)-3-(2-benzyloxy-5-iodophenyl)-2(S)-tert-butoxycarbonylaminopropionic acid [(−)-6A] as example

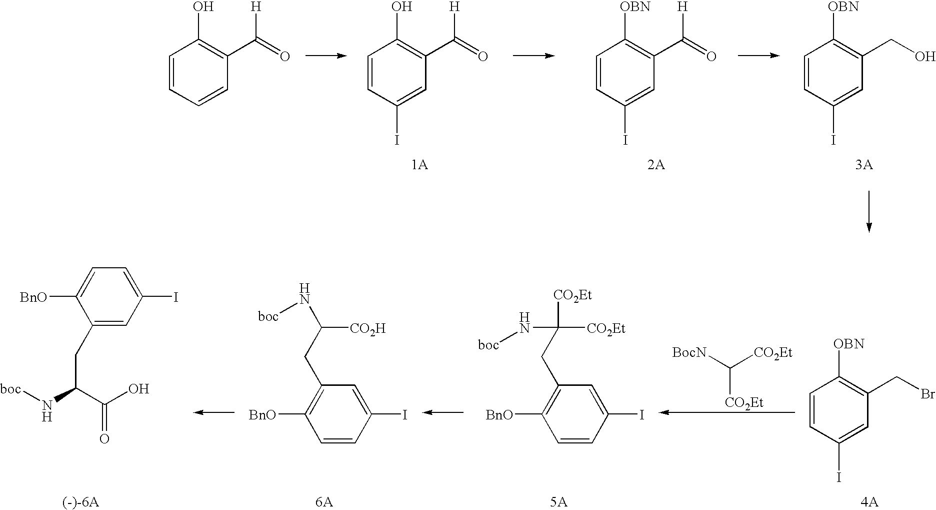

### Synthesis of protected biphenyl-bisamino acids with 2(S)-trimethylsilanylethyl 2(S)-benzyloxycarbonylamino-3-[4,4′-bisbenzyloxy-3′-(2(S)benzyloxycarbonyl-2(S)-tert-butoxycarbonylaminoethyl)biphenyl-3-yl]propionate (12A) as example

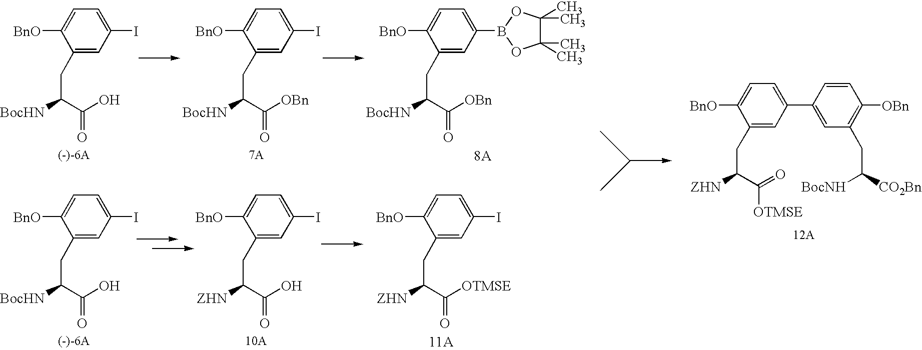

### Synthesis of protected hydroxy ornithine derivatives with 5-benzyloxycarbonylamino-2(S)-tert-butoxycarbonylamino-4(R)-(tert-butyldimethylsilyloxy)pentanoic acid (14A) as example

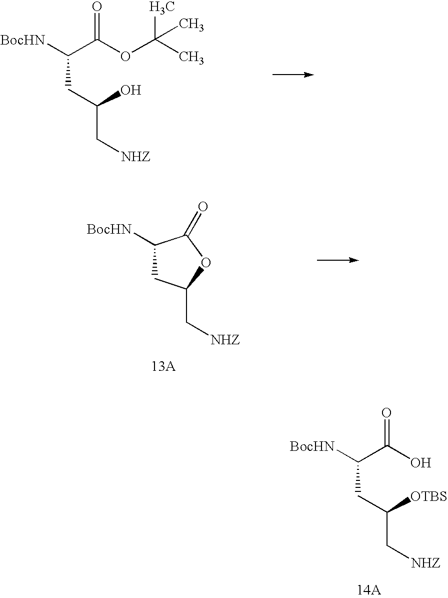 Synthesis of Exemplary Embodiments 1 and 2: 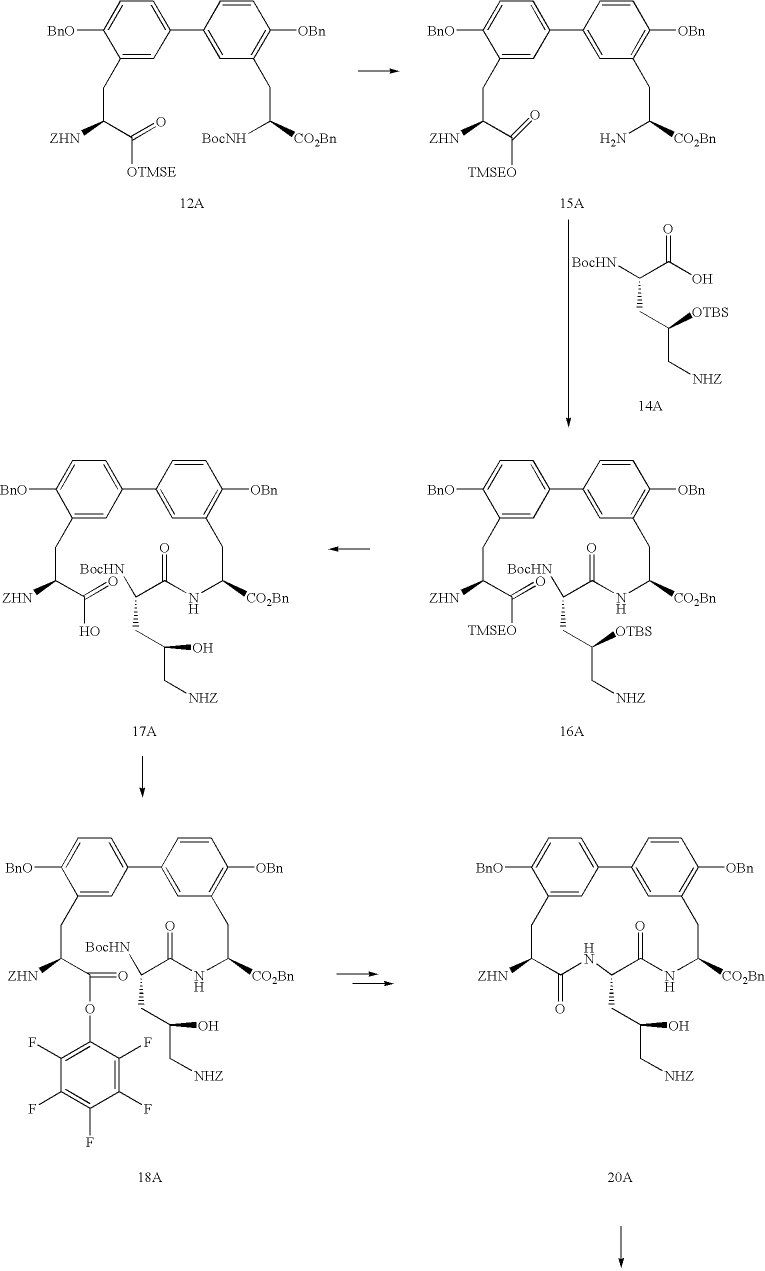 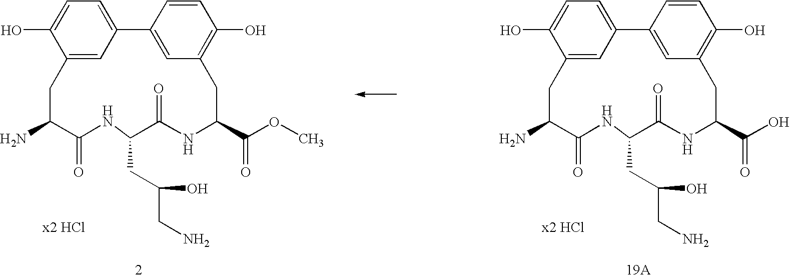
Starting Compounds and Exemplary Embodiments

### Example 1A

### 2-Hydroxy-5-iodobenzaldehyde

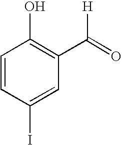

A solution of 250 g (1.54 mol) of iodine chloride in 600 ml of anhydrous dichloromethane is added dropwise over the course of 2 h to a solution of 188 g (1.54 mol) of salicylaldehyde in 1 l of anhydrous dichloromethane in a heat-dried flask under argon. After stirring at RT for 3 days, a saturated aqueous sodium sulfite solution is added with vigorous stirring. The organic phase is separated off, washed once with water and saturated aqueous sodium chloride solution and dried over sodium sulfate. The solvent is evaporated and the residue is recrystallized from ethyl acetate. 216 g (57% of theory) of the product are obtained.

LC-MS (ESI, Method 10): m/z=246 (M-H)⁻.

¹H-NMR (400 MHz, CDCl₃): δ=6.7 (d, 1H), 7.77 (dd, 1H), 7.85 (d, 1H), 9.83 (s, 1H), 10.95 (s, 1H).

### Example 2A

### 2-Benzyloxy-5-iodobenzaldehyde

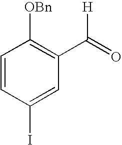

67.2 g (0.48 mol) of potassium carbonate are added to a solution of 100 g (0.40 mol) of 2-hydroxy-5-iodobenzaldehyde (Example 1A) in 1.5 l of dimethylformamide and, after a few minutes, 51 ml (0.44 mol) of benzyl chloride are added. The reaction mixture is stirred under reflux at 120° C. for 24 h. After stirring at RT for a further 24 h and addition of 1.5 l of water, a solid crystallizes out. The precipitate is filtered off with suction, washed twice with water and dried in vacuo. The solid is recrystallized from 230 ml of ethanol. 122.9 g (90% of theory) of the product are obtained.

LC-MS (ESI, Method 10): m/z 338 (M+H)⁺.

¹H-NMR (400 MHz, CDCl₃): δ=5.18 (s, 2H), 6.84 (d, 1H), 7.33-7.45 (m, 5H), 7.78 (dd, 1H), 8.12 (d, 1H), 10.4 (s, 1H).

### Example 3A

### (2-Benzyloxy-5-iodophenyl)methanol

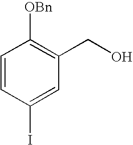

100 ml of 1 M diisobutylaluminum hydride solution in dichloromethane are added to a solution, cooled to 0° C., of 33.98 g (100.5 mmol) of 2-benzyloxy-5-iodobenzaldehyde (Example 2A) in 200 ml of dichloromethane. After stirring at 0° C. for 2 h, a saturated potassium sodium tartrate solution is added while cooling (highly exothermic reaction), and the reaction mixture is stirred for a further 2 h. After separation of the phases, the organic phase is washed twice with water and once with saturated aqueous sodium chloride solution and dried over sodium sulfate. The solvent is evaporated off in vacuo. 31.8 g (93% of theory) of the product are obtained.

¹H-NMR (400 MHz, CDCl₃): δ=2.17 (t, 1H), 4.68 (d, 2H), 5.1 (s, 2H), 6.72 (d, 1H), 7.32-7.42 (m, 5H), 7.54 (dd, 1H), 7.63 (d, 1H).

### Example 4A

### 1-Benzyloxy-2-bromomethyl-4-iodobenzene

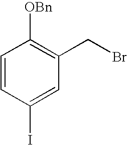

3.3 ml (35 mmol) of phosphorus tribromide are added dropwise to a solution of 35 g (103 mmol) of (2-benzyloxy-5-iodophenyl)methanol (Example 3A) in 350 ml of toluene at 40° C. The temperature of the reaction mixture is raised to 100° C. over the course of 15 min and the mixture is stirred at this temperature for a further 10 min. After cooling the two phases are separated. The organic phase is washed twice with distilled water and once with saturated aqueous sodium chloride solution. The organic phase is dried over sodium sulfate and evaporated. The yield amounts to 41 g (99% of theory).

¹H-NMR (300 MHz, CDCl₃): δ=4.45 (s, 2H), 5.06 (s, 2H), 7.30 (m, 8H).

### Example 5A

### Diethyl 2-(2-benzyloxy-5-iodobenzyl)-2-tert-butoxycarbonylaminomalonate

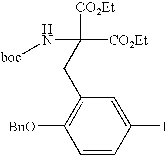

41 g (101.7 mmol) of 1-benzyloxy-2-bromomethyl-4-iodobenzene (Example 4A) are added to a solution of 28 g (101.7 mmol) of diethyl 2-[N-(tert-butoxycarbonyl)amino]malonate and 7.9 ml (101.7 mmol) of sodium ethoxide in 300 ml of ethanol. After stirring at RT for 3 h, the precipitated product is filtered off with suction. After drying in vacuo, 55 g (90% of theory) of product are isolated.

1H-NMR (400 MHz, CDCl₃): δ=1.12 (t, 6H), 1.46 (s, 9H), 3.68 (s, 2H), 3.8-3.9 (m, 2H), 4.15-4.25 (m, 2H), 5.0 (s, 2H), 5.7 (s, 1H), 6.58 (d, 1H), 7.28-7.4 (m, 6H), 7.4 (dd, 1H).

### Example 6A

### (+/−)-3-(2-Benzyloxy-5-iodophenyl)-2-tert-butoxycarbonylaminopropionic acid

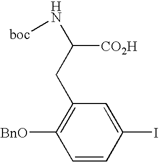

400 ml of 1 N sodium hydroxide solution are added to a suspension of 58 g (97 mmol) of diethyl 2-(2-benzyloxy-5-iodobenzyl)-2-tert-butoxycarbonyl-aminomalonate (Example 5A) in 800 ml of a mixture of ethanol and water (7:3). After 3 h under reflux and after cooling to room temperature, the pH of the reaction mixture is adjusted to about pH 2 with conc. hydrochloric acid. The reaction mixture is evaporated. The residue is taken up in MTBE and water. The aqueous phase is extracted three times with MTBE. The combined organic phases are dried over sodium sulfate, filtered and concentrated. Drying in vacuo results in 47 g (97% of theory) of the product.

¹H-NMR (400 MHz, DMSO): δ=1.32 (s, 9H), 2.68 (dd, 1H), 3.18 (dd, 1H), 4.25 (m, 1H), 5.15 (s, 2H), 6.88 (d, 1H), 7.08 (d, 1H), 7.30-7.40 (m, 3H), 7.45-7.55 (m, 3H).

### Example (−)-6A

### 3-(2-Benzyloxy-5-iodophenyl)-2(S)-tert-butoxycarbonylaminopropionic acid

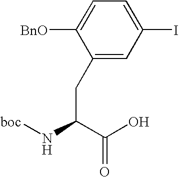

The racemate from Example 6A [(+/−)-3-(2-benzyloxy-5-iodophenyl)-2(S)-tert-butoxycarbonylaminopropionic acid] is separated on a chiral stationary silica gel phase based on the selector from poly(N-methacryloyl-L-leucine dicyclopropylmethylamide) using an i-hexane/ethyl acetate mixture as eluent. The enantiomer eluted first (98.9% ee) is dextrorotatory in, dichloromethane ([α]²_{D}: +3.00; c=0.54, dichloromethane) and corresponds to the (R) enantiomer Example (+)-6A, as was determined by single-crystal X-ray structural analysis. The purity of the second, levorotatory enantiomer Example (−)-6A, i.e. the (S) enantiomer, is >99% ee.

### Example 7A

### Benzyl 3-(2-benzyloxy-5-iodophenyl)-2(S)-tert-butoxycarbonylaminopropionate

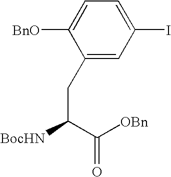 then heated to 80° C. under a gentle stream of argon and after 6 h is cooled again. The mixture is purified by column chromatography on silica gel (mobile phase: dichloromethane). DMSO residues present are removed by Kugelrohr distillation. The residue is again purified by column chromatography on silica gel (mobile phase: cyclohexane:ethyl acetate 4:1).

Yield: 8.15 g (79% of theory).

HPLC (method 3): Rₜ=6.26 min.

LC-MS (method 6): Rₜ=5.93 and 6.09 min.

MS (E): m/z=588 (M+H)⁺.

¹H-NMR (200 MHz, CDCl₃): δ=1.26 (s, 6H), 1.33 (s, 9H), 1.36 (s, 614), 2.91-3.10 (m, 1H), 3.12-3.28 (m, 1H), 4.49-4.68 (m, 1H), 5.05 (dd, 2H), 5.11 (dd, 2H), 5.30 (d, 1H), 6.90 (d, 1H), 7.27-7.37 (m, 7H), 7.38-7.42 (m, 3H), 7.55-7.62 (m, 1H), 7.67 (dd, 1H).

### Example 9A

### 2(S)-Amino-3-(2-benzyloxy-5-iodophenyl)propionic acid hydrochloride

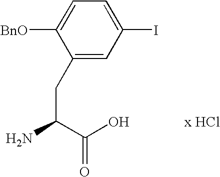

12 g (24.13 mmol) of 3-(2-benzyloxy-5-iodophenyl)-2(S)-tert-butoxycarbonylaminopropionic acid [Example (−)-6A] are put under argon into 60 ml of 4 M hydrochloric acid solution in dioxane and stirred at RT for 2 h. The reaction solution is concentrated and dried under high vacuum.

Yield: 10.47 g (100% of theory).

HPLC (Method 3): Rₜ=4.10 min.

MS (EI): m/z=398 (M+H−HCl)⁺.

¹H-NMR (200 MHz, CDCl₃): δ=3.17-3.31 (m, 1H), 3.33-3.47 (m, 1H), 4.22 (t, 1H), 5.13 (s, 2H), 6.69 (d, 1H), 7.24-7.40 (m, 2H), 7.41-7.45 (m, 2H), 7.48 (d, 1H), 7.52 (d, 1H), 7.60 (d, 1H), 8.66 (br.s, 2H).

### Example 10A

### 2(S)-Benzyloxycarbonylamino-3-(2-benzyloxy-5-iodophenyl)propionic acid

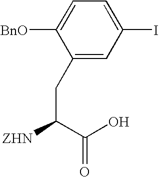

9.25 ml (53.09 mol) of N,N-diisopropylethylamine are added to a solution of 10.46 g (24.13 mmol) of 2(S)-amino-3-(2-benzyloxy-5-iodophenyl)propionic acid hydrochloride (Example 9A) in DMF. 6.615 g (26.54 mmol) of N-(benzyloxycarbonyl)succinimide (Z-OSuc) are added thereto. The resulting solution is stirred overnight and then evaporated in vacuo. The residue is taken up in dichloromethane and extracted twice each with 0.1 N hydrochloric acid solution and saturated aqueous sodium chloride solution. The organic phase is dried, filtered and concentrated. The mixture is purified by column chromatography on silica gel (mobile phase: cyclohexane/diethyl ether 9:1 to 8:2).

Yield: 8.30 g (65% of theory)

HPLC (method 3): Rₜ=5.01 min.

MS (EI): m/z=532 (M+H)⁺.

¹H-NMR (200 MHz, DMSO): δ=3.14-3.3 (m, 2H), 4.25-4.45 (m, 1H), 4.97 (s, 2H), 5.14 (s, 2H), 6.88 (d, 1H), 7.20-7.56 (m, 12H), 7.62 (d, 1H), 12.73 (br.s, 1H).

### Example 11A

### (2-Trimethylsilyl)ethyl 2(S)-benzyloxycarbonylamino-3-(2-benzyloxy-5-iodophenyl)propionate

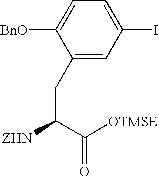

8.35 g (15.7 mmol) of 2(S)-benzyloxycarbonylamino-3-(2-benzyloxy-5-iodophenyl)propionic acid (Example 10A) are introduced into 150 ml of THF, and 2.14 g (18.07 mmol) of 2-trimethylsilylethanol and 250 mg (2.04 mmol) of 4-dimethylaminopyridine are added. The mixture is cooled to 0°, and 2.38 g (2.95 ml, 18.86 mmol) of N,N′-diisopropylcarbodiimide dissolved in 40 ml of THF are added. The mixture is stirred at RT overnight and evaporated in vacuo for working up. The residue is taken up in dichloromethane and extracted twice each with 0.1 N hydrochloric acid solution and saturated aqueous sodium chloride solution. The organic phase is dried, filtered and concentrated. The mixture is purified by column chromatography (silica gel, mobile phase: cyclohexane/diethyl ether 9:1 to 8:2).

Yield: 8.2 g (83% of theory).

HPLC (method 3): Rₜ=6.42 min

MS (EI): m/z=532 (M+H)⁺.

¹H-NMR (300 MHz, CDCl₃): δ=0.01 (s, 9H), 0.88 (t, 2H), 2.96 (dd, 1H), 3.13 (dd, 1H), 4.04-4.17 (m, 2H), 4.51-4.62 (m, 1H), 4.95-5.05 (m, 4H), 5.44 (d, 1H), 6.64 (d, 1H), 7.25-7.33 (m, 7H), 7.37 (dd, 4H), 7.45 (dd, 1H).

### Example 12A

### 2-(Trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate

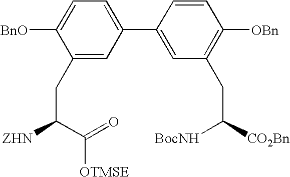 Method A:

45.8 mg (0.05 mmol) of bis(diphenylphosphino)ferrocenepalladium(II) chloride (PdCl₂(dppf)) and 0.325 g (1.0 mmol) of cesium carbonate are added to a solution of 0.316 g (0.5 mmol) of (2-trimethylsilyl)ethyl 2(S)-benzyloxycarbonylamino-3-(2-benzyloxy-5-iodophenyl)propionate (Example 1A) in 2.5 ml of degassed DMF under argon at RT. The reaction mixture is heated to 40° C. Over the course of 30 min, a solution of 0.294 g (0.5 mmol) of benzyl 3-[2-benzyloxy-5-(4,4,5,5-tetramethyl-[1,3,2]dioxaborolan-2-yl)phenyl]-2(S)-tert-butoxycarbonylaminopropionate (Example 8A) in 2.5 ml of degassed DMF is added dropwise. The reaction mixture is stirred at 40° C. for 4 h and at 50° C. for a further 2 h. The solvent is evaporated and the residue is taken up in ethyl acetate. The organic phase is extracted twice with water, dried over sodium sulfate and concentrated. The crude product is purified by chromatography on silica gel with dichloromethane/ethyl acetate (30/1). 0.320 g (66% of theory) of the product is obtained.

Method B:

A solution of 6.99 g (11.06 mmol) of (2-trimethylsilyl)ethyl 2(S)-benzyloxycarbonylamino-3-(2-benzyloxy-5-iodophenyl)propionate (Example 11A) and 6.50 g (11.06 mmol) of benzyl 3-[2-benzyloxy-5-(4,4,5,5-tetramethyl-[1,3,2]dioxaborolan-2-yl)phenyl]-2(S)-tert-butoxycarbonylaminopropionate (Example 8A) in 40 ml of DMF is degassed by passing argon through (about 30 min.). Then 812 mg (1.11 mmol) of bis(diphenylphosphino)ferrocenepalladium(II) chloride (PdCl₂(dppf)) and 7.21 g (22.13 mmol) of cesium carbonate are added thereto. A gentle stream of argon is passed over the reaction mixture, which is heated at 80° C. for 2.5 h. The mixture is cooled and purified by column chromatography on silica gel (mobile phase: cyclohexane/ethyl acetate 7:3). Before evaporation to dryness is complete, diisopropyl ether is added to the mixture. The resulting crystals are filtered off with suction and dried under high vacuum.

Yield: 6.54 g (61% of theory).

HPLC (method 3): Rₜ=7.65 min

MS (EI): m/z=987 (M+Na), 965 (M+H)⁺.

¹H-NMR (200 MHz, CDCl₃): δ=0.00 (s, 9H), 0.90 (t, 2H), 1.37 (s, 9H), 3.02-3.35 (m, 4H) 4.06-4.25 (m, 2H), 4.55-4.73 (m, 2H), 4.98-5.18 (m, 8H), 5.40 (d, 1H), 5.63 (d, 1H), 6.88-7.00 (m, 2H), 7.19-7.39 (m, 20H), 7.42-7.53 (m, 4H).

### Example 13A

### N^{a}-(tert-Butoxycarbonyl)-N^{ε}(benzyloxycarbonyl)-(2S,4R)-hydroxyornithine lactone

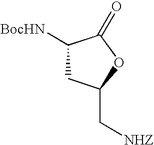

A solution of 7.60 g (17.3 mmol) of tert-butyl 5-benzyloxycarbonylamino-2(S)-tert-butoxycarbonylamino-4(R)-hydroxypentanoate (preparation described in Org. Lett. 2001, 3, 20, 3153-3155) in 516 ml of dichloromethane and 516 ml of trifluoroacetic acid is stirred at RT for 2 h. The solvent is evaporated. The remaining crude product is dissolved in 2.6 l of anhydrous methanol and, while stirring at 0° C., 6.3 g (28.8 mmol) of di-tert-butyl dicarbonate and 7.3 ml (52.43 mmol) of triethylamine are added. After 15 h, the reaction solution is evaporated and the residue is taken up in 1 l of ethyl acetate. After the phases have been separated, the organic phase is extracted twice with a 5% strength citric acid solution, twice with water and once with saturated aqueous sodium chloride solution, dried over sodium sulfate and concentrated. The crude product is purified by chromatography on silica gel with toluene/acetone (5/1). 4.92 g (78% of theory) of the product are obtained.

LC-HR-FT-ICR-MS (method 13): calc. for C₁₈H₂₈N₃O₆ (M+NH₄)⁺ 382.19726 found 382.19703.

¹H-NMR (400 MHz, CDCl₃): δ=1.45 (s, 9H), 2.3-2.4 (m, 1H), 2.45-2.55 (m, 1H), 3.3-3.4 (m, 1H), 3.5-3.6 (m, 1H), 4.17-4.28 (m, 1H), 4.74.8 (m, 1H), 5.0-5.15 (m, 4H), 7.3-7.4 (m, 5H).

### Example 14A

### 5-Benzyloxycarbonylamino-2(S)-tert-butoxycarbonylamino-4(R)-(tert-butyldimethylsilanyloxy)pentanoic acid

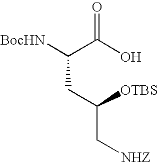 Method A:

2 ml of 1 M sodium hydroxide solution are added to a solution of 0.73 g (2 mmol) of N^{a}(tert-butoxycarbonyl)-N^{ε}(benzyloxycarbonyl)-(2S,4R)-hydroxyomithine lactone (13A) in 50 ml of 1,4-dioxane at 0° C. The reaction solution is stirred for 2 h and then evaporated. The residue is taken up in 50 ml of dichloromethane. 1.12 ml (8 mmol) of triethylamine are added to this solution and, after a short time, 1.38 ml (6 mmol) of tert-butyldimethylsilyl trifluoromethanesulfonate are added dropwise. After stirring at RT for 3 h, the reaction mixture is diluted with dichloromethane. The organic phase is washed with 1 N sodium bicarbonate solution, dried over sodium

### Example 15A

### 2-(Trimethylsilyl)ethyl 3-[3′-(2(S)-amino-2-benzyloxycarbonylethyl)-4,4′-bisbenzyloxybiphenyl-3-yl]-2(S)-benzyloxycarbonylaminopropionate hydrochloride

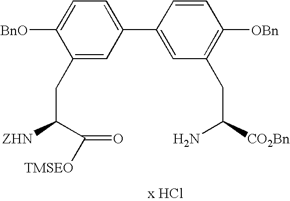

50 ml of a 4 M hydrochloric acid/dioxane solution are added over the course of about 20 min to a solution, cooled to 0° C., of 2.65 g (2.75 mmol) of 2-(trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate (Example 12A) in 50 ml of anhydrous dioxane. After stirring for 3 h, the reaction solution is evaporated and dried under high vacuum.

Yield: 100% of theory.

HPLC (Method 3): Rₜ=5.96 min

MS (EI): m/z=865 (M+H)⁺.

### Example 16A

### Benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate

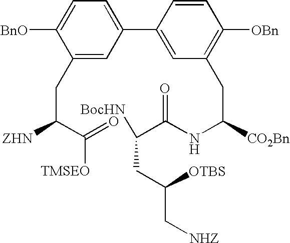

0.219 g (0.58 mmol) of HATU and 0.082 g (0.63 mmol) of N,N-diisopropylethylamine are added to a solution, cooled to 0° C., of 0.520 g (0.58 mmol) of (2-trimethylsilyl)ethyl 3-[3′-(2(S)-amino-2-benzyloxycarbonylethyl)-4,4′-bisbenzyloxybiphenyl-3-yl]-2(S)-benzyloxycarbonylaminopropionate hydrochloride (Example 15A) and 0.287 g (0.58 mmol) of 5-benzyloxycarbonylamino-2(S)-tert-butoxycarbonylamino-4(R)-(tert-butyldimethylsilyloxy)pentanoic acid (Example 14A) in 7.3 ml of anhydrous DMF. After stirring at 0° C. for 30 min, an additional 0.164 g (1.26 mmol) of N,N-diisopropylethylamine is added. The reaction mixture is stirred at RT for 15 h. The solvent is then evaporated, and the residue is taken up in ethyl acetate. The organic phase is washed three times with water and once with saturated aqueous sodium chloride solution, dried over sodium sulfate and concentrated. The crude product is purified by chromatography on silica gel with dichloromethane/ethyl acetate (gradient 30/1→20/1→10/1). 533 mg (66% of theory) of the product are obtained.

LC-MS (ESI, method 12): m/z 1342 (M+H)⁺, 1365 (M+Na)⁺.

### Example 17A

### 2(S)-Benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid

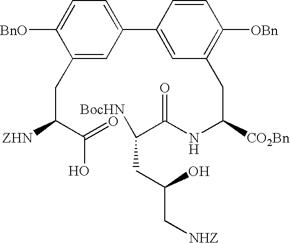 Method A:

0.80 ml of a 1.0 M solution of tetrabutylammonium fluoride in THF is added to a solution of 0.360 g (0.27 mmol) of benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate (Example 16A) in 22.5 ml of anhydrous DMF. After stirring at RT for 1 h, the reaction mixture is cooled to 0° C., and water is added. After addition of ethyl acetate, the phases are separated. The organic phase is washed with a 1.0 M solution of potassium bisulfate, dried over sodium sulfate and evaporated. 0.331 g of the crude product is obtained. The crude product is reacted without further purification.

LC-MS (ESI, method 10): m/z=1129 (M+H)⁺.

LC-HR-FT-ICR-MS: calc. for C₆₅H₆₉N₄O₁₄ (M+H)⁺ 1129.48048 found 1129.48123.

Method B:

1.8 ml of 1N tetrabutylammonium fluoride in THF are added dropwise to a solution of 800 mg (0.6 mmol) of benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate (Example 16A) in 26 ml of absolute DMF at RT. After 25 min at RT, the mixture is cooled to 0° C. and a large amount of ice-water is added. Ethyl acetate and some 1N hydrochloric acid solution are immediately added. The organic phase is dried with magnesium sulfate, concentrated and dried under high vacuum for 1 h. The crude product is reacted without further purification.

### Example 18A

### Benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate

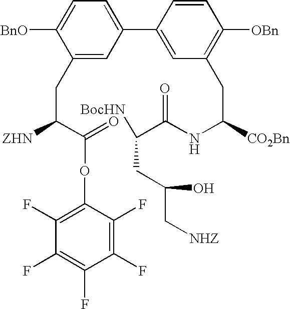 Method A:

90 mg of pentafluorophenol (0.49 mmol), dissolved in a little dichloromethane, and 1.1 mg of 4-dimethylaminopyridine (10 μM) and 19.4 mg (0.10 mmol) of EDC are added to a solution, cooled to −25° C., of 104 mg (92 μmol) of 2(S)-benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid (Example 17A) in 3 ml of dichloromethane under argon. After stirring for 15 h, the reaction mixture is concentrated. The crude product is reacted without further purification.

LC-MS (ESI, method 11): m/z 1317 (M+Na)⁺, 1295 (M+H)⁺.

LC-HR-FT-ICR-MS: calc. for C₇₁H₆₈F₅N₄O₁₄ (M+H)⁺ 1295.46467 found 1295.46430.

Method B:

691 mg (crude mixture, approx. 0.6 mmol) of 2(S)-benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid (Example 17A) are introduced into 25 ml of dichloromethane, and 547.6 mg (2.98 mmol) of pentafluorophenol, dissolved in 6 ml of dichloromethane, are added. 7.3 mg (0.06 mmol) of DMAP are added, and the mixture is cooled to −25° C. (ethanol/carbon dioxide bath). At −25° C., 148 mg (0.774 mmol) of EDC are added. The mixture slowly warms to RT overnight. The reaction mixture is concentrated in vacuo and briefly dried under high vacuum. The crude product is reacted without further purification.

### Example 19A

### 14(S)-Amino-11(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride

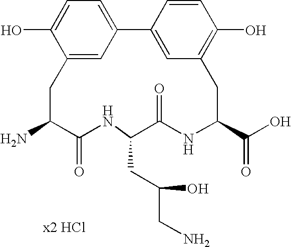 Method A:

A solution of 10 mg (9.9 μM) of benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]henicosa-1(19),2,4,6(21),16(20),17-hexaene-8(S)-carboxylate (Example 20A) and 50 μl of formic acid in 10 ml of ethanol is vigorously stirred in the presence of 10 mg of Pd/C under hydrogen at atmospheric pressure for 16 h. The reaction solution is evaporated, and the residue is taken up in 1 N hydrochloric acid solution and filtered. The crude product is purified on an RP 18 cartridge with acetonitrile/water. 2 mg (42.8% of theory) of the product are obtained.

Method B:

200 mg (0.20 mmol) of benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]henicosa-1(19),2,4,6(21),16(20),17-hexaene-8(S)-carboxylate (Example 20A) are put into 220 ml of an acetic acid/water/ethanol 4:1:1 mixture (ethanol can be replaced by THF). 73 mg of 10% palladium/carbon (10% Pd/C) are added, and then hydrogenation is carried out under atmospheric pressure for 15 h. The reaction mixture is filtered through prewashed kieselguhr, and the filtrate is concentrated in vacuo. The residue is mixed with 4.95 ml of 0.1 N aqueous hydrochloric acid and concentrated. The residue is stirred with 10 ml of diethyl ether and decantered. The remaining solid is dried under high vacuum.

Yield: 103 mg (95% of theory).

HPLC (method 3): Rₜ=3.04 min;

LC-MS (method 6): Rₜ=0.38 min

MS (EI): m/z=473 (M+H)⁺.

¹H-NMR (400 MHz, D₂O): δ=2.06-2.20 (m, 1H), 2.74-2.89 (m, 1H), 2.94-3.05 (m, 1H), 3.12-3.25 (m, 2H), 3.53 (d, 1H), 3.61-3.72 (m, 1H), 3.97-4.07 (m, 1H), 4.53 (s, 1H), 4.61 (d, 1H), 4.76-4.91 (m, 12H), 7.01-7.05 (m, 2H), 7.07 (s, 1H), 7.40-7.45 (m, 2H), 7.51 (d, 1H).

### Example 20A

### Benzyl 5,17-bisbenzyloxy-14(S)-benzyloxycarbonylamino-11(S)-(3-benzyloxycarbonylamino-2(R)hydroxypropyl)-10,13-dioxo-9,12-diazatricyclo[14.3.1.1^{2,6}]-henicosa-1(19),2,4,6(21),16(20),17-hexaene-8(S)-carboxylate

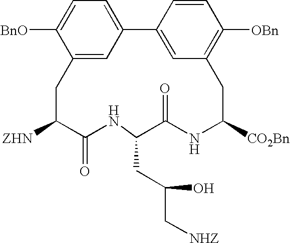 Method A:

4 ml of a 4 M hydrochloric acid solution in 1,4-dioxane are added to a solution of 119.3 mg of benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate (Example 18A) in 2.7 ml of 1,4-dioxane. Until the reaction is complete, a further 1.5 ml of 4 M hydrochloric acid solution in 1,4-dioxane is added. The reaction solution is evaporated and codistilled with chloroform twice. The crude product (LC-HR-FT-ICR-MS, Method 13: calc. for C₆₆H₆₀F₅N₄O₁₂ (M+H)⁺ 1195.41224, found 1195.41419) is dissolved in 100 ml of chloroform and added dropwise over the course of 3 h to a very efficiently stirred suspension of 200 ml of chloroform and 100 ml of saturated aqueous sodium bicarbonate solution. The reaction mixture is vigorously stirred for 2 h. After the two phases have been separated, the aqueous phase is extracted with chloroform. The combined organic phases are washed with 5% strength aqueous citric acid solution, dried over magnesium sulfate and evaporated to dryness. The crude product is washed with acetonitrile and dried under high vacuum.

Yield: 60.5 mg (65% of theory)

LC-MS (ESI, method 11): m/z=1011 (M+H)⁺.

Method B:

About 0.595 mmol of benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate (Example 18A) are dissolved in 8 ml of dioxane and then, at 0° C., 16 ml of 4 N hydrochloric acid solution in dioxane are added dropwise. After 45 min, 6 ml of 4 N hydrochloric acid solution in dioxane are again added, and after 15 min a further 8 ml are added. The mixture is stirred at 0° C. for 30 min before the reaction solution is concentrated under mild conditions, codistilled with chloroform (twice) and briefly dried under high vacuum. The crude product (732 mg, 0.59 mmol) is dissolved in 1000 ml of chloroform, and a solution of 6 ml of triethylamine in 50 ml of chloroform is added dropwise. The mixture is stirred at RT overnight. The mixture is worked up by evaporating under mild conditions in vacuo and stirring the residue in acetonitrile. The resulting crystals are filtered off with suction, washed with acetonitrile and dried under high vacuum.

Yield: 360 mg (60% of theory).

MS (EI): m/z=1011 (M+H)⁺

HPLC (method 3): Rₜ=5.59 min.

¹H-NMR (400 MHz, d₆-DMSO): δ=1.52-1.65 (m, 1H), 1.73-1.84 (m, 1H), 2.82-3.01 (m, 3H), 3.02-3.11 (m, 1H), 3.46 (s, 1H), 3.57-3.68 (m, 1H), 4.47-4.56 (m, 1H), 4.64-4.71 (m, 1H), 4.73-4.85 (m, 2H), 4.88-5.00 (m, 4H), 5.09 (s, 2H), 5.14-5.20 (m, 4H), 6.29 (d, 1H), 7.00-7.11 (m, 4H), 7.21-7.40 (m, 20H), 7.41-7.48 (m, 9H), 8.77 (d, 1H), 8.87 (d, 1H).

### Example 21A

### Benzyl 2(S)-tert-butoxycarbonylamino-5-nitro-4-oxopentanoate

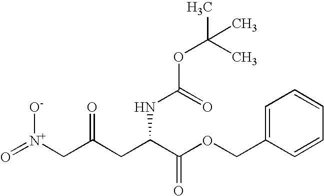

A solution A of 10 g (30.9 mmol) of 2(S)-tert-butoxycarbonylaminosuccinic acid 1-benzyl ester and 5.27 g (32.5 mmol) of 1,1′-carbonyldiimidazole in 100 ml of tetrahydrofuran is stirred at RT for 5 h. 18.8 g (30.9 mmol) of nitromethane are added dropwise to a solution B of 3.2 g (34.2 mmol) of potassium tert-butoxide in 100 ml of tetrahydrofuran at 0° C. Solution B is stirred while warming to RT, and then solution A is added dropwise at RT. The resulting mixture is stirred at RT for 16 h and adjusted to pH 2 with 20% strength hydrochloric acid. The solvent is evaporated. The remaining crude product is taken up in ethyl acetate/water. After separation of the phases, the organic phase is extracted twice with water, dried over sodium sulfate and concentrated. 13 g (99% of theory) of the product are obtained.

MS (ESI): m/z=334 (M+H)⁺

¹H-NMR (300 MHz, d₆-DMSO): δ=1.37 (s, 9H), 2.91 (m, 1H), 3.13 (m, 1H), 4.44 (m, 1H), 5.12 (s, 2H), 5.81 (m, 2H), 7.2-7.5 (m, 5H).

### Example 22A

### Benzyl 2(S)-tert-butoxycarbonylamino-4(R)-hydroxy-5-nitropentanoate

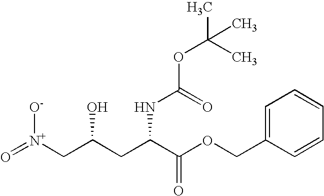

A solution of 11.3 g (30.8 mmol) of benzyl 2(S)-tert-butoxycarbonylamino-5-nitro-4-oxopentanoate in 300 ml of tetrahydrofuran is cooled to −78° C., 30.8 ml of a 1M solution of L-Selectrid® in tetrahydrofuran are added dropwise, and the mixture is stirred at −78° C. for 1 h. After warming to RT, saturated ammonium chloride solution is cautiously added to the solution. The reaction solution is concentrated, and the residue is taken up in water and ethyl acetate. The aqueous phase is extracted three times with ethyl acetate. The combined organic phases are dried over sodium sulphate and evaporated. The crude product is prepurified on silica gel 60 (mobile phase: cyclohexane/ethyl acetate 10/1), and the collected fractions are concentrated and stirred with cyclohexane/ethyl acetate 5/1. The remaining crystals are filtered off with suction and dried. 2.34 g (21% of theory) of the desired diastereomer are obtained. Chromatographic separation of the mother liquor on Lichrospher Diol 10 μm (mobile phase: ethanol/isohexane 5/95) results in a further 0.8 g (6.7% of theory) of the product.

MS (ESI): m/z=369 (M+H)⁺

¹H-NMR (300 MHz, d₆-DMSO): δ=1.38 (s, 9H), 1.77 (m, 1H), 1.97 (m, 1H), 4.10-4.44 (m, 3H), 4.67 (m, 1H), 5.12 (m, 2H), 5.49 (d, 1H), 7.25-7.45 (m, 5H).

### Example 23A

### Benzyl 2(S)-[S-benzyloxycarbonylamino-2(S)-tert-butoxycarbonylaminopentanoylamino]-3-{4,4′-bisbenzyloxy-3′-[2(S)-benzyloxycarbonylamino-2-(2-trimethylsilylethoxycarbonyl)ethyl]biphenyl-3-yl}propionate

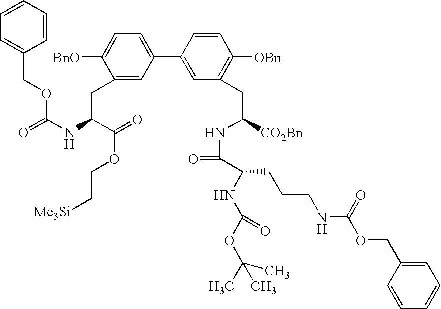

Preparation takes place in analogy to Example 16A from 0.47 g (0.51 mmol) of the compound from Example 15A and 0.19 g (0.51 mmol) of N_{α}-boc-N_{δ}-Z-L-ornithine with 0.19 g (0.51 mmol) of HATU and 0.35 ml (1.65 mmol) of N,N-diisopropylethylamine in 5.55 ml of dry DMF.

Yield: 0.58 g (92% of theory)

LC-MS (method 18): Rₜ=3.46 min

MS: m/z=1212 (M+H)⁺

### Example 24A

### 2(S)-Benzyloxycarbonylamino-3-{4,4′-bisbenzyloxy-3′-[2(S)-benzyloxycarbonyl-2-(5-benzyloxycarbonylamino)-2(S)-tert-butoxycarbonylaminopentanoylamino)-ethyl]biphenyl-3-yl}-propionic acid

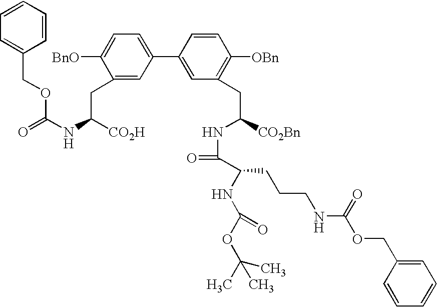

Preparation takes place in analogy to Example 17A from 0.82 g (0.68 mmol) of the compound from Example 23A with 2 equivalents (1.3 ml) of tetrabutylammonium fluoride (1M in THF) in 30 ml of dry DMF.

Yield: 772 mg (94% of theory)

LC-MS (method 19): Rₜ=1.62 min

MS: m/z=1112 (M+H)⁺

### Example 25A

### Benzyl 2(S)-(5-benzyloxycarbonylamino-2(S)-tert-butoxycarbonylaminopentanoylamino)-3-[4,4′-bisbenzyloxy-3′-(2(S)-benzyloxycarbonylamino-2-pentafluorophenyloxycarbonylethyl)biphenyl-3-yl]propionate

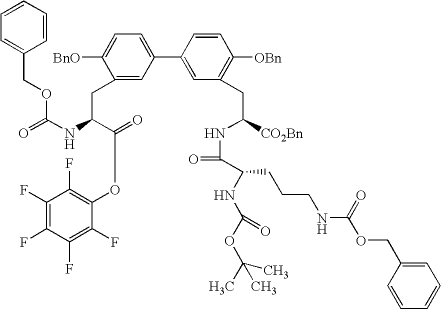

Preparation takes place in analogy to Example 18A (method A) from 422 mg (0.38 mmol) of the compound from Example 24A and 349 mg (1.9 mmol) of pentafluorophenol with 80 mg (0.42 mmol) of EDC and 4.63 mg (0.04 mmol) of DMAP in 4 ml of dichloromethane.

Yield: 502 mg (95% of theory)

LC-MS (method 19): Rₜ=3.13 min

MS: m/z=1278 (M+H)⁺

### Example 26A

### Benzyl 2(S)-(5-benzyloxycarbonylamino-2(S)-aminopentanoylamino)-3-[4,4′-bisbenzyloxy-3′-(2-(S)-benzyloxycarbonylamino-2-pentafluorophenyloxycarbonylethyl)biphenyl-3-yl]propionate hydrochloride

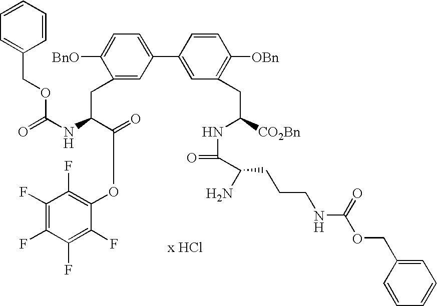

5 ml of a 4M solution of hydrogen chloride in dioxane are added to 215 mg (0.17 mmol) of the compound from Example 25A while stirring in an ice bath. The mixture is stirred for one hour and evaporated to constant weight in vacuo.

Yield: 200 mg (92% of theory)

LC-MS (method 19): Rₜ=4.25 min

MS: m/z=1178 (M+H)⁺

### Example 27A

### Benzyl 5,17-bisbenzyloxy-14(S)-benzyloxycarbonylamino-11(S)-(3-benzyloxycarbonylaminopropyl)-10,13-dioxo-9,12-diazatricyclo[14.3.1.1^{2,6}]henicosa-1(19),2,4,6(21),16(20),17-hexaene-8(S)-carboxylate

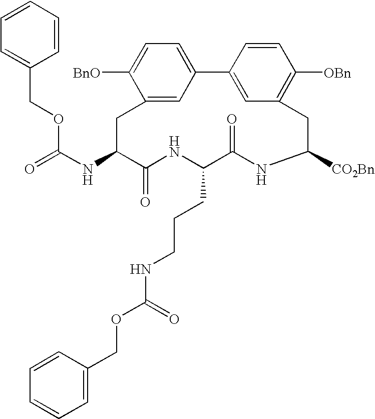

1.35 g (0.91 mmol) of the compound from Example 26A are introduced into 3 l of chloroform and, while stirring vigorously, 2.54 ml (18.2 mmol) of triethylamine in 50 ml of chloroform are added at RT over the course of 20 min. The mixture is left to stir overnight and evaporated to dryness in vacuo. The residue is stirred with 5 ml of acetonitrile and filtered, and the residue is dried to constant weight.

Yield: 890 mg (93% of theory)

LC-MS (method 19): Rₜ=5.10 min

MS: m/z=994 (M+H)⁺

### Example 28A

### (8S,11S,14S)-14-Amino-11-(3-aminopropyl)-5,17-dihydroxy-10,13-dioxo-9,12-diazatricyclo[14.3.1.1^{2,6}]henicosa-1(20),2(21),3,5,6,18-hexaene-8-carboxylic acid dihydrochloride

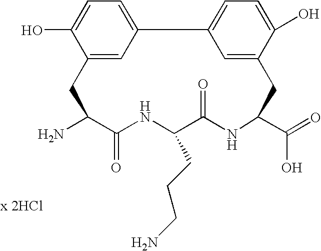

50 mg (0.05 mmol) of the compound from Example 27A are suspended in 50 ml of glacial acetic acid/water/ethanol (4/1/1), 30 mg of Pd/C (10%) catalyst are added, and the mixture is hydrogenated at RT for 20 hours. After removal of the catalyst by filtration through kieselguhr, the filtrate is evaporated to dryness in vacuo and, while stirring, 2.5 ml of 0.1N hydrochloric acid are added. The mixture is evaporated to dryness in vacuo and dried to constant weight.

Yield: 17 mg (63% of theory)

TLC (methanol/dichloromethane/25% ammonia=5/3/2): Rₜ=0.6

LC-MS (method 9): Rₜ=0.28 min

MS: m/z=457 (M+H)⁺

### Example 29A

### (8S,11S,14S)-14-[(tert-Butoxycarbonyl)amino-11-[3-[(tert-butoxycarbonyl)-amino]propyl}-5,17-dihydroxy-10,13-dioxo-9,12-diazatricyclo[14.3.1.1^{2,6}]henicosa-1(20),2(21),3,5,16,18-hexaene-8-carboxylic acid

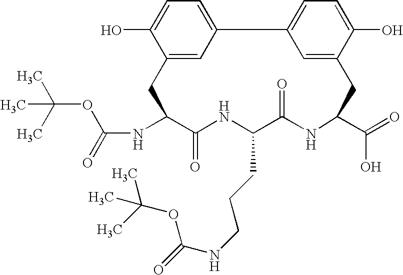

225 mg (0.42 mmol) of the compound from Example 28A are dissolved in 2.25 ml of water and 2.25 ml of 1 N sodium hydroxide solution and cooled in an ice bath and, while stirring, 278 mg (1.27 mmol) of di-tert-butyl dicarbonate are added. After the addition, the mixture is briefly heated to 30° C. and left to react further at RT overnight. The mixture is acidified to about pH=5 with 0.1 N hydrochloric acid and cautiously evaporated to dryness and RT in vacuo. The residue is stirred with diethyl ether, filtered and dried to constant weight.

Yield: 259 mg (93% of theory)

LC-MS (method 18): Rₜ=1.96 min.

MS: m/z=656 (M+H)⁺

### Example 30A

### 2-(Benzyloxy)-N-(tert-butoxycarbonyl)iodo-N-methyl-L-phenylalanine

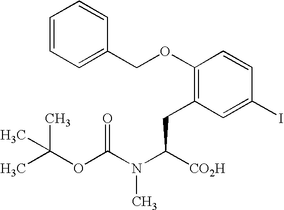

Under an argon atmosphere, 500 mg (1 mmol) of the compound from Example 6A are dissolved in 20 ml of THF, 90.5 mg (3.02 mmol) of sodium hydride and 0.51 ml (1141.6 mg; 8.04 mmol) of methyl iodide (80% pure) are added, and the mixture is stirred at room temperature overnight. It is diluted with 25 ml of ethyl acetate and 25 ml of water and adjusted to pH 9 with 0.1N hydrochloric acid. The mixture is concentrated to a small volume in vacuo. 10 ml of ethyl acetate and 10 ml of water are added, the mixture is shaken vigorously, and the organic phase is separated off. Drying with sodium sulfate and concentration in vacuo result in 140 mg of product (19% of theory). The aqueous phase is acidified (pH=3) and extracted three times with 20 ml of ethyl acetate. Concentration in vacuo and drying in vacuo result in 351 mg of product (68% of theory).

LC-MS (method 17): Rₜ=3.9 min

MS (EI): m/z=511 (M+H)⁺

### Example 31A

### Benzyl 2-(benzyloxy)-N-(tert-butoxycarbonyl)-5-iodo-N-methyl-L-phenylalaninate

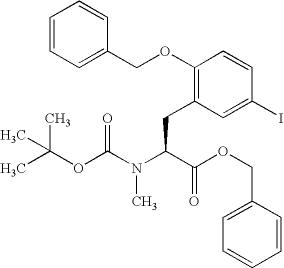

Preparation takes place in analogy to Example 7A from 350 mg (0.68 mmol) of the compound from Example 30A, 8.29 mg (0.07 mmol) of DMAP, 148 mg (1.37 mmol) of benzyl alcohol and 157.46 mg (0.82 mmol) of EDC in 3 ml of acetonitrile.

Yield: 382 mg (93% of theory)

LC-MS (method 17): Rₜ=4.8 min

MS (EI): ml=601 (M+H)⁺

### Example 32A

### Benzyl 2-(benzyloxy)-N-(tert-butoxycarbonyl)-N-methyl-5-(4,4,5,5-tetramethyl-1,3,2-dioxaborolan-2-yl)-L-phenylalaninate

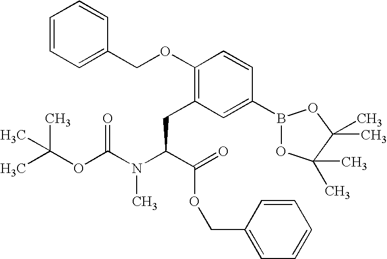

In analogy to Example 8A, 380 mg (0.63 mmol) of the compound from Example 31A are introduced into 4 ml of DMF in a heat-dried flask and, while stirring at room temperature, 184.5 mg (0.73 mmol) of 4,4,4′,4′,5,5,5′,5′-octamethyl-2,2′-bi-1,3,2-dioxaborolane, 186 mg (1.9 mmol) of potassium acetate and 23.15 mg (0.03 mmol) of bis(diphenylphosphino)ferrocenepalladium(II) chloride are added. Reaction is allowed to take place at 80° C. for 4 h. The product is obtained after workup and chromatography (silica gel 60, mobile phase: cyclohexane/ethyl acetate=4/1).

Yield: 196 mg

LC-MS (method 17): Rₜ=4.9 min

MS (EI): m/z=601 (M+H)⁺

### Example 33A

### 2-(Trimethylsilyl)ethyl 2(S)-benzyloxycarbonylamino-3-[4,4′-bisbenzyloxy-3′-(2(S)-benzyloxycarbonyl-(2-tert-butoxycarbonyl-2-methyl)aminoethyl)biphenyl-3-yl]propionate

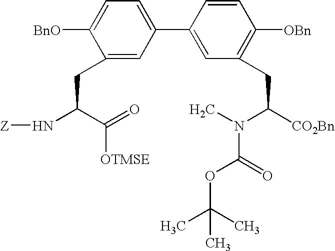

Preparation takes place in analogy to Example 12A (method B) from 190 mg (0.32 mmol) of the compound from Example 32A, 199.5 mg (0.32 mmol) of the compound from Example 1A, 195.5 mg (0.63 mmol) of cesium carbonate and 23.15 mg (0.03 mmol) of bis(diphenylphosphino)ferrocenepalladium(II) chloride in 1.5 ml of DMF under an argon atmosphere.

Yield: 212 mg (66% of theory)

LC-MS (method 22): Rₜ=4.86 min

MS (EI): m/z 978 (M+H)⁺

### Example 34A

### 2-(Trimethylsilyl)ethyl 2(S)-benzyloxycarbonylamino-3-[4,4′-bisbenzyloxy-3′-(2(S)-benzyloxycarbonyl-2-methylaminoethylbiphenyl-3-yl]propionate hydrochloride

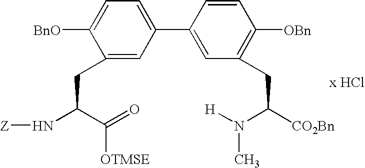

Preparation takes place in analogy to Example 15A from 930 mg (0.95 mmol) of the compound from Example 33A and 22.14 ml of a 4M solution of hydrogen chloride in dioxane, in 15 ml of dioxane.

Yield: 915 mg (78% of theory)

LC-MS (method 22): Rₜ=2.53 min

MS (EI): m/z=878 (M+H)⁺

### Example 35A

### Benzyl 2(S)-{Methyl-[5-benzyloxycarbonylamino-2(S)-tert-butoxycarbonylamino-4(R)-(tert-butyldimethylsilyloxy)pentanoyl]amino}-3-{4,4′-bisbenzyloxy-3′-[2(S)-benzyloxycarbonylamino-2-(2-trimethylsilylethoxycarbonyl)ethyl]-biphenyl-3-yl}propionate

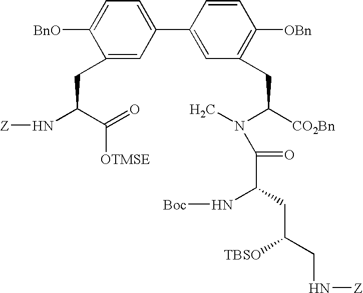

Preparation takes place in analogy to Example 16A from 922 mg (1.01 mmol) of the compound from Example 34A, 0.5 g (1.01 mmol) of the compound from Example 14A, 421 mg (1.11 mmol) of HATU and 0.7 ml (518 mg; 3.27 mmol) of DIPEA in 4.2 ml of DMF.

Yield: 703 mg (51% of theory)

LC-MS (method 16): Rₜ=3.17 min

MS (EI): m/z=1356 (M+H)⁺

### Example 36A

### 2(S)-Benzyloxycarbonylamino-3-{4,4′-bisbenzyloxy-3′-[2(S)-benzyloxycarbonyl-2-{methyl-(5-benzyloxycarbonylamino-2(S)-tert-butoxycarbonylamino-4(R)-hydroxypentanoyl)amino}ethyl]biphenyl-3-yl}propionic acid

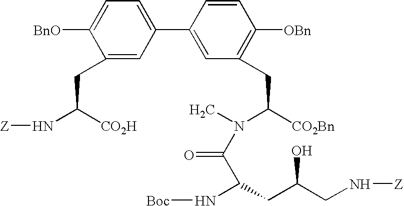

Preparation takes place in analogy to Example 17A from 360 mg (0.27 mmol) of the compound from Example 35A and 0.8 ml (3 equivalents) of 1M tetrabutylammonium fluoride solution (THF) in 20 ml of DMF.

Yield: 159 mg (53% of theory)

LC-MS (method 21): Rₜ=3.19 min

MS (EI): m/z 1142 (M+H)⁺

### Example 37A

### Benzyl 2(S)-[methyl-(5-benzyloxycarbonylamino)-2(S)-tert-butoxycarbonylamino-4(R)-hydroxypentanoyl]amino-3-[4,4′-bisbenzyloxy-3′-(2(S)-benzyloxycarbonylamino-2-pentafluorophenyloxycarbonylethyl)biphenyl-3-yl]propionate

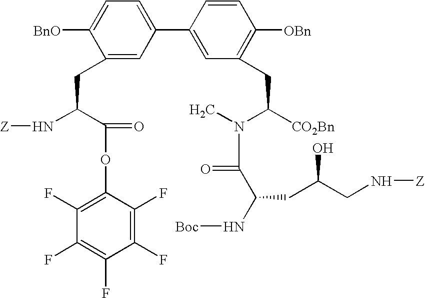

Preparation takes place in analogy to Example 18A (method A) from 330 mg (0.29 mmol) of the compound from Example 36A, 265.6 mg (1.44 mmol) of pentafluorophenol, 3.53 mg (0.03 mmol) of DMAP and 60.87 mg (0.32 mmol) of EDC in 10 ml of dichloromethane.

Yield: 271 mg (69% of theory)

LC-MS (method 21): Rₜ=3.38 min

MS (EI): m/z=1308 (M+H)⁺

### Example 38A

### Benzyl 2(S)-[methyl-(5-benzyloxycarbonylamino)-2(S)-amino-4(R)-hydroxypentanoyl]amino-3-[4,4′-bisbenzyloxy-3′-(2(S)-benzyloxycarbonylamino-2-pentafluorophenyloxycarbonylethyl)biphenyl-3-yl]propionate hydrochloride

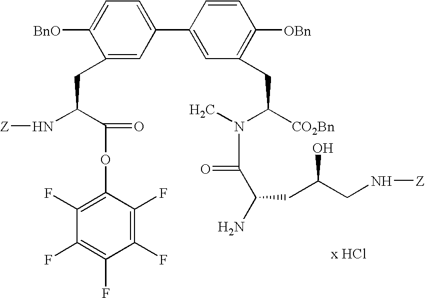

130 mg (0.1 mmol) of the compound from Example 37A are dissolved in 0.5 ml of dioxane, and 5 ml of a 4M solution of hydrogen chloride in dioxane are cautiously added (ice bath). After 30 minutes, reaction is allowed to continue at room temperature for a further 2 h. The mixture is evaporated to dryness in vacuo and dried to constant weight under high vacuum.

Yield: 130 mg (70% of theory)

LC-MS (method 15): Rₜ=2.68 min

MS (E): m/z=1208 (M+H)⁺

### Example 39A

### Benzyl(8S,11S,14S)-5,17-bis(benzyloxy)-14-{[(benzyloxy)carbonyl]amino}-11-((2R)-3-{[(benzyloxy)carbonyl]amino}-2-hydroxypropyl-9-methyl-10,13-dioxo-9,12-diazatricyclo[14.3.1.1^{2,6}]henicosa-1(20),2(21),3,5,16,18-hexaene-8-carboxylate

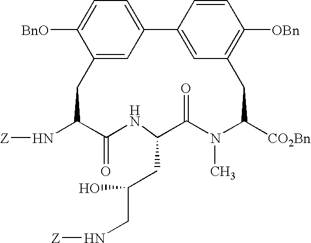

130 mg (0.1 mmol) of the compound from Example 38A are introduced into 220 ml of dry chloroform. While stirring at room temperature, 23 ml (20 eq.) of triethylamine in 5 ml of dichloromethane are added over the course of 20 minutes. The mixture is stirred overnight. It is then evaporated to dryness in vacuo. The residue is stirred with acetonitrile. Drying of the residue results in 44 mg of product. Further product (30 mg) is obtained from the mother liquor by RP-HPLC.

Yield: 74 mg (69% of theory)

LC-MS (method 15): Rₜ=3.13 min

MS (EI): m/z=1024 (M+H)⁺

### Example 40A

### (8S,11S,14S)-14-Amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaenecarboxylic acid di(trifluoroacetate)

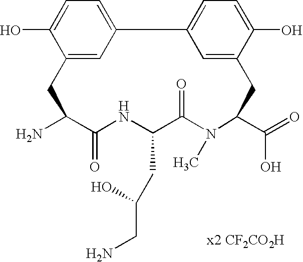

33 mg (0.032 mmol) of the compound from Example 39A are cautiously treated with dilute trifluoracetic acid. The resulting clear solution is subsequently lyophilized.

Yield: 23 mg (quantitative)

LC-MS (method 15): Rₜ=0.92 min

MS (EI): m/z=486 (M+H)⁺

### Example 41A

### (8S,11S,14S)-5,17-Bis(benzyloxy)-14-{[benzyloxycarbonyl]amino}-11-(2R)-3-{[benzyloxycarbonyl]amino}-2-hydroxypropyl-9-methyl-10,13-dioxo-9,12-diazatricyclo[14.3.1.1^{2,6}]henicosa-1(20),2(21),3,5,16,18-hexaene-8-carboxylic acid

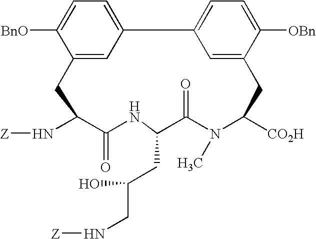

37 mg (0.04 mmol) of the compound from Example 39A are dissolved in 2 ml of THF, 0.14 ml of 1N lithium hydroxide solution is added, and the mixture is stirred at room temperature for 3 h. It is then acidified with 1N hydrochloric acid and evaporated to dryness under high vacuum.

Yield: 33 mg (71% of theory)

LC-MS (Method 21): Rₜ=2.90 min

MS (EI): m/z=934 (M+H)⁺

Examples 42A to 48A listed in the following table are prepared from the appropriate starting compounds in analogy to the methods of Examples 35A to 41A detailed above:

| | | prepared | |
|---|---|---|---|
| Example | | in analogy | |
| No. | Structure | to | Analytical data |
| 42A | 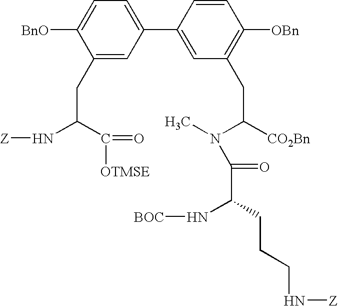 | 35A | LC-MS (method 22): Rₜ =4.85 min. MS (EI): m/z = 1226 (M + H)⁺ |
| 43A | 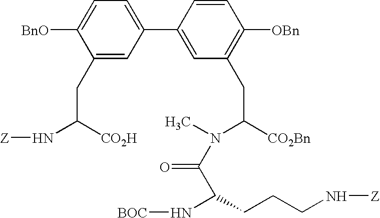 | 36A | LC-MS (method 22): Rₜ =2.04 min. MS (EI): m/z = 1126 (M + H)⁺ |
| 44A | 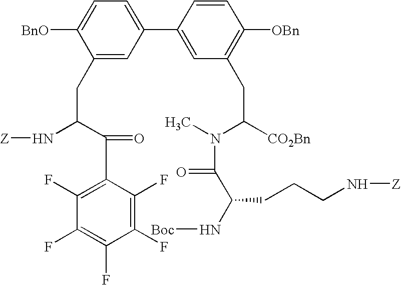 | 37A | LC-MS (method 22): Rₜ =3.79 min. MS (EI): m/z = 1292 (M + H)⁺ |
| 45A | 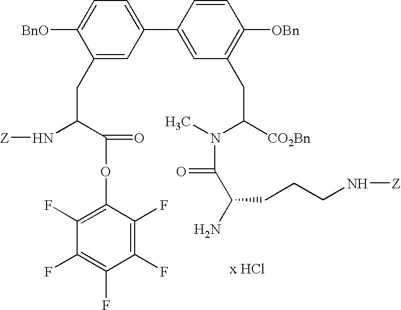 | 38A | LC-MS (method 22): Rₜ =3.72 min. MS (EI): m/z = 1192 (M + H)⁺ |
| 46A | 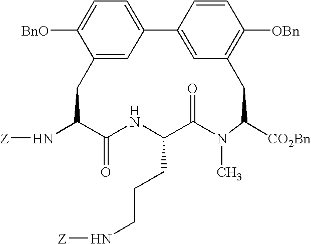 | 39A | LC-MS (method 22): Rₜ =4.39 min. MS (EI): m/z = 1008 (M + H)⁺ |
| 47A | 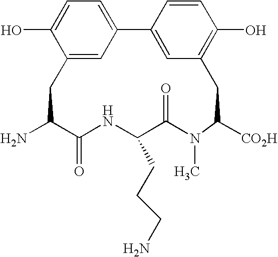 | 40A | LC-MS (method 21): Rₜ =0.53 min. MS (EI): m/z = 470 (M + H)⁺ |
| 48A | 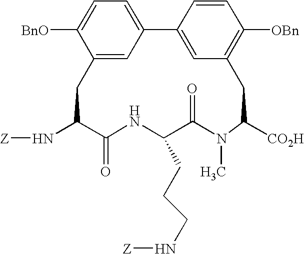 | 41A | LC-MS (method 23): Rₜ =3.64 min. MS (EI): m/z = 918 (M + H)⁺ |

### Example 49A

### 2-[(tert-Butoxycarbonyl)amino]ethyl(8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaene-8-carboxylate

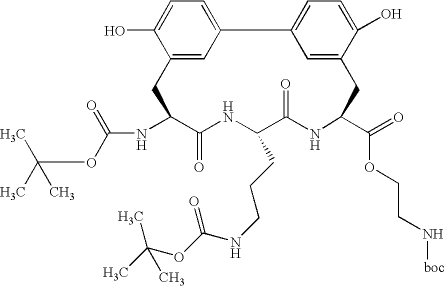

133 mg (0.2 mmol) of the compound from Example 29A are introduced into 2 ml of dichloromethane, mixed with 97.9 mg (0.61 mmol) of tert-butyl 2-hydroxyethylcarbamate and 12.37 mg (0.1 mmol) of DMAP and cooled to 0° C. 47.3 mg (0.37 mmol) of DIC are added, and the mixture is stirred at 0° C. for 1 h and then at room temperature for 4 h. The mixture is subsequently evaporated to dryness in vacuo, and the residue is separated by HPLC.

Yield: 18 mg (11% of theory)

LC-MS (method 24): Rₜ=3.8 min.

MS (EI): m/z=799 (M+H)⁺

### Example 50A

### (8S,11S,14S)-5,17-Bis(benzyloxy)-14-{1(benzyloxy)carbonyl]amino}-11-(3-{[(benzyloxy)carbonyl]amino}propyl)-10,13-dioxo-9,12-diazatricyclo[14.3.1.1^{2,6}]henicosa-1(20),2(21),3,5,16,18-hexaene-8-carboxylic acid

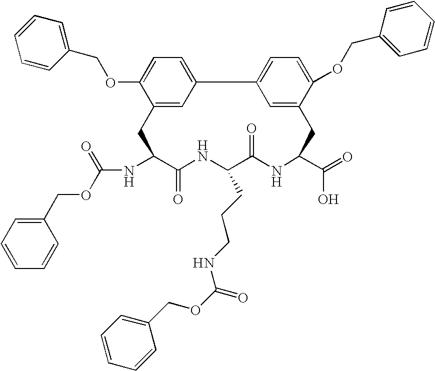

200 mg (0.2 mmol) of the compound from Example 27A are introduced into 8 ml of THF and 4 ml of DMF and, while stirring, 0.8 ml of a 1 M aqueous lithium hydroxide solution (4 equivalents) is added. A gel is produced after stirring at room temperature for 2 h. 0.8 ml of 1 N hydrochloric acid and also some water are added. The mixture is then evaporated to dryness in vacuo and stirred with water, and the precipitate is filtered off and dried.

Yield: 140 mg (77% of theory)

LC-MS (method 18): Rₜ=2.83 min.

MS (EI): m/z 904 (M+H)⁺

### Example 51A

### 2-(Benzyloxy)-2-oxoethyl(8S,11S,14S)-5,17-bis(benzyloxy)-14-{[(benzyloxy)-carbonyl]amino}-11-(3-{[(benzyloxy)carbonyl]amino}propyl)-10,13-dioxo-9,12-diazatricyclo[14.3.1.1^{2,6}]henicosa-1(20),2(21),3,5,16,18-hexaene-8-carboxylate

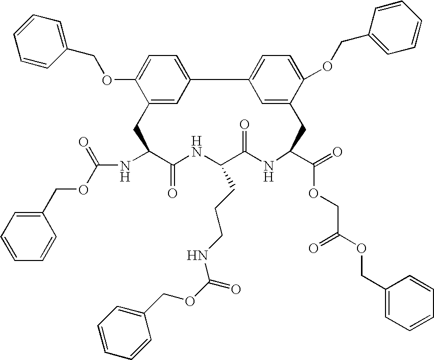

20 mg (0.02 mmol) of the compound from Example 50A are suspended in 2 ml of DMF and heated (oil bath temperature 50° C.). After 50 minutes, 9.16 mg (0.07 mmol) of finely powdered potassium carbonate are added to the fine suspension. After stirring for 1 h, 10.12 mg (0.04 mmol) of benzyl bromoacetate are added, and the reaction is allowed to take place while stirring at a bath temperature of 50-60° C. overnight. After cooling, water is added, and the precipitate is stirred. The product is obtained after filtration and drying.

Yield: 11 mg (36% of theory)

LC-MS (method 24): Rₜ=4.2 min.

MS (EI): m/z 1052 (M+H)⁺

### Example 52A

### (8S,11S,14S)-14-[(tert-Butoxycarbonyl)amino]-11-{3-[(tert-butoxycarbonyl)-amino]propyl}-5,17-dihydroxy-10,13-dioxo-9,12-diazatricyclo[14.3.1.1^{2,6}]henicosa-1(20),2(21),3,5,16,18-hexaene-8-carboxylic acid

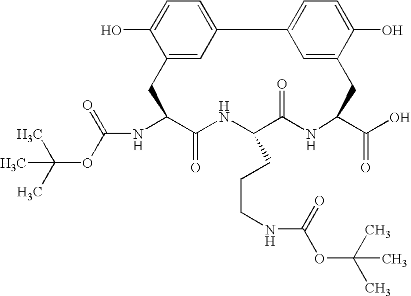

90 mg (0.16 mmol) of the compound from Example 40A are dissolved in 2.5 ml of water, mixed with 85.3 mg (0.8 mmol) of sodium carbonate and cooled in an ice bath, and 105.3 mg (0.48 mmol) of di-(tert-butyl) dicarbonate in 1.2 ml of methanol are added. The mixture is stirred at room temperature overnight, concentrated to a small volume in vacuo and acidified to pH=2 with 1 N hydrochloric acid. The resulting precipitate is filtered off and dried.

Yield: 89 mg (73% of theory)

LC-MS (method 21): Rₜ=1.8 min.

MS (EI): m/z=686 (M+H)⁺

### Example 53A

### 2-[(tert-Butoxycarbonyl)amino]ethyl(8S,11S,14S)-14-[(tert-butoxycarbonyl)-amino]-11-{(2S)-3-[(tert-butoxycarbonyl)amino]-2-hydroxypropyl}-5,17-dihydroxy-10,13-dioxo-9,12-diazatricyclo[14.3.1.1^{2,6}]henicosa-1(20),2(21),3,5,16,18 hexaene-8-carboxylate

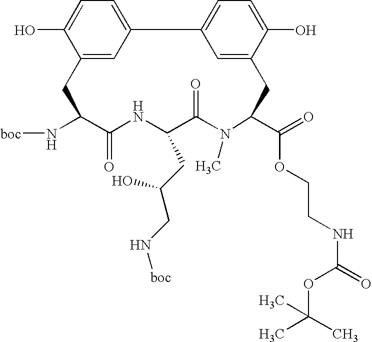

Preparation takes place in analogy to Example 49A from 20 mg (0.03 mmol) of the compound from Example 52A and 9.4 mg (0.06 mmol) of tert-butyl 2-hydroxyethylcarbonate with 6.7 mg (0.03 mmol) of EDC in 1 ml of acetonitrile.

Yield: 4 mg (15% of theory)

LC-MS (method 21): Rₜ=2.19 min.

MS (EI): m/z=829 (M+H)⁺

### Exemplary Embodiments

### Example 1

### Methyl(8S,11S,14S)-14-amino-11-[(2R)-3-amino-2-hydroxypropyl]-5,17-dihydroxy-10,13-dioxo-9,12-diazatricyclo[14.3.1.1^{2,6}]henicosa-1(20),2(21),3,5,16,18-hexaene-8-carboxylate dihydrochloride

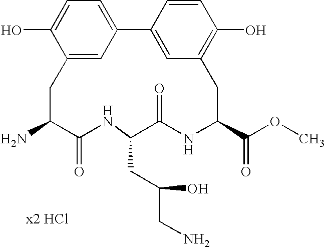

2.2 mg (4.0 μmol) of 14(S)-amino-11(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride (Example 19A) are dissolved in dry methanol (analytical grade, 1.2 ml) under a protective argon gas atmosphere. While stirring vigorously at RT, 50 μl (0.2 μmol) of a 4M dioxane/hydrogen chloride solution are added dropwise. The mixture is stirred at RT, and the reaction is followed by HPLC chromatography. Complete conversion is reached after about one to two days. The reaction mixture is evaporated in vacuo and dried under high vacuum, resulting in the product in a yield of 4.4 mg (97% of theory).

HPLC/UV-Vis (method 14): Rₜ=3.6 min.

λₘₐₓ (qualitative)=204 nm (s), 269 (m), 285 (sh)

(H₂O/acetonitrile+0.01% TFA [7:3]).

LC-MS (ESI): m/z (%)=487 (35) [M+H]⁺, 285 (45), 265 (100).

LC-HR-FT-ICR-MS calc. for C₂₄H₃₁N₄O₇ [M+H]⁺ 487.2187 found 487.2189.

### Example 2

### Ethyl(8S,11S,14S)-14-amino-11-[(2R)-3-amino-2-hydroxypropyl]-5,17-dihydroxy-10,13-dioxo-9,12-diazatricyclo[14.3.1.1^{2,6}]henicosa-1(20),2(21),3,5,16,18-hexaene-8-carboxylate dihydrochloride

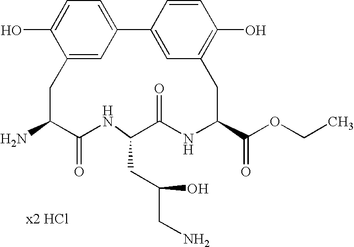

1.6 mg (2.9 μmol) of 14(S)-amino-11(S)-(3-amino-2(R)-hydroxy-propyl)-5,17-dihydroxy-10,13-dioxo-9,12-diaza-tricyclo[14.3.1.1^{2,6}]henicosa-1(19),2,4,6(21),16(20),17-hexaene-8(S)-carboxylic acid dihydrochloride (Example 19A) are dissolved in absolute ethanol (1.0 ml) under a protective argon gas atmosphere. While stirring vigorously at RT, 40 μl (0.15 μmol) of a 4M dioxane/hydrogen chloride solution are added dropwise. The mixture is stirred at room temperature and the reaction is followed by HPLC chromatography. Complete conversion is reached after about one to two days. The reaction mixture is concentrated in vacuo and dried under high vacuum. The product is obtained in a yield of 1.4 mg (85% of theory).

HPLC/UV-Vis (method 14): Rₜ=3.9 min.,

λₘₐₓ (qualitative)=206 nm (s), 270 (m), 285 (sh)

(H₂O/acetonitrile+0.01% TFA [7:3]).

LC-MS (ESI): m/z (%)=501 (90) [M+H]⁺.

LC-HR-FT-ICR-MS calc. for C₂₅H₃₃N₄O₇ [M+H]⁺ 501.2344 found 501.2347.

### Example 3

### Methyl(8S,11S,14S)-14-amino-11-(3-aminopropyl)-5,17-dihydroxy-10,13-dioxo-9,12-diazatricyclo[14.3.1.1^{2,6}]-henicosa-1(20),2(21),3,15,16,18-hexaene-8-carboxylate dihydrochloride

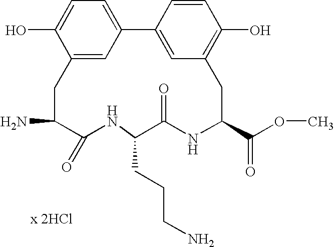

30 mg (0.057 mmol) of the compound from Example 28A are introduced into 15 ml of methanol under an argon atmosphere, mixed with 0.5 ml of 4M dioxane/hydrogen chloride solution and stirred at room temperature for 3 hours. The mixture is then evaporated to dryness in vacuo, and the residue is dried to constant weight.

Yield: 25.2 mg (820% of theory)

LC-MS (method 23): Rₜ=2.9 min.

MS (E): m/z=470 [M+H]⁺

### Example 4

### 2-Methyl(8S,11S,14S)-14-amino-11-(3-aminopropyl)-5,17-dihydroxy-10,13-dioxo-9,12-diazatricyclo[14.3.1.1^{2,6}]-henicosa-1(20),2(21),3,15,16,18-hexaene-8-carboxylate trihydrochloride

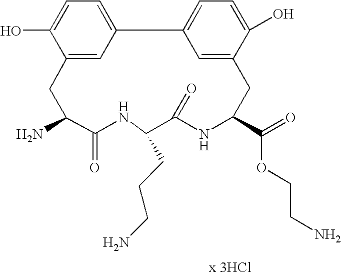

9 mg (0.01 mmol) of the compound from Example 49A are cooled in an ice bath, and 1 ml of 4 M dioxane/hydrogen chloride solution is added. A precipitate separates out after stirring for two hours. It is filtered off and dried to constant weight under high vacuum.

Yield: 7 mg (73% of theory)

LC-MS (method 20): Rₜ=0.27 min.

MS (E): m/z=499 [M+H]⁺

### Example 5

### Isobutyl(8S,11S,14S)-14-amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxylate dihydrochloride

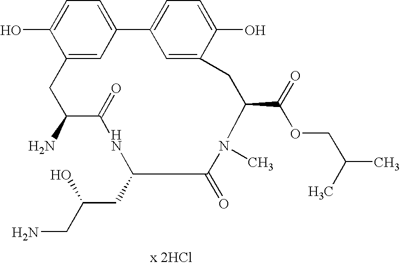

10 mg (0.02 mmol) of the free acid (Example 19A) are suspended in 1.25 ml of isobutanol, and 10 drops of dioxane/4M hydrogen chloride solution are added. Reaction is allowed to take place with stirring at RT for 3 days. The mixture is evaporated to dryness in vacuo, and the residue is dried to constant weight.

Yield: 11 mg (90% of theory)

LC-MS (method 21): Rₜ=1.14 min.

MS (EI): m/z=542 (M+H)⁺

### Example 6

### Methyl(8S,11S,14S)-14-amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxylate bis(trifluoroacetate)

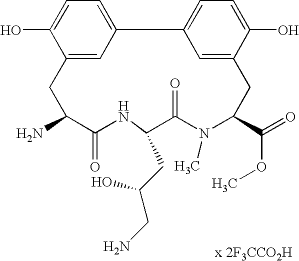

On hydrogenation of 65 mg (0.06 mmol) of the compound from Example 39A in analogy to Example 40A, the free acid is treated with a little methanol in the presence of hydrogen chloride and evaporated to dryness in vacuo at a bath temperature of 50° C. This results in the methyl ester. Addition of a few drops of trifluoroacetic acid is followed by evaporation to dryness in vacuo and drying to constant weight.

Yield: 46.2 mg (quantitative)

LC-MS (method 18): Rₜ=1.19 min.

MS (EI): m/z 500 (M+H)⁺

### Example 7

### Methyl(8S,11S,14S)-14-amino-11-(3-aminopropyl)-5,17-dihydroxy-9-methyl-10,13-dioxo-9,12-diazatricyclo[14.3.1.1^{2,6}]henicosa-1(20),2(21),3,5,16,18-hexaene-8-carboxylate dihydrochloride

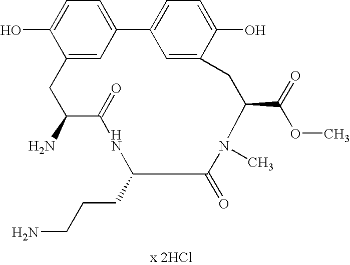

Preparation takes place in analogy to Example 5 from 1.2 mg of the compound from Example 40A with 0.3 ml of absolute methanol and 3 drops of 4M dioxane/hydrogen chloride solution.

Yield: 1.2 mg (quantitative)

LC-MS (method 21): Rₜ=0.89 min.

MS (EI): m/z=484 (M+H)⁺

### Example 8

### ({[(8S,11S,14S)-14-Amino-11-(3-aminopropyl)-5,17-dihydroxy-10,13-dioxo-9,12-diazatricyclo[14.3.1.1^{2,6}]henicosa-1(20),2(21),3,5,16,18-hexaen-8-yl]carbonyl}-oxy)carboxylic acid dihydrochloride

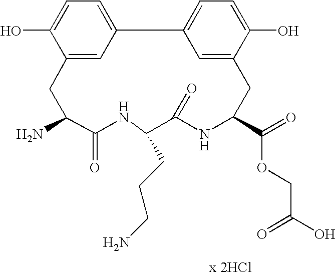

11 mg (0.01 mmol) of the compound 51A are suspended in ethanol/water/glacial acetic acid, mixed with 6 mg of Pd/C (10%) catalyst and hydrogenated at RT and atmospheric pressure for 6 h. The mixture is evaporated to dryness in vacuo, and the desired product is stirred with acetonitrile and precipitated with 0.1 N hydrochloric acid. It is dissolved in a little methanol, and the product is separated on a thick-layer plate, mobile phase: glacial acetic acid/ethanol/water=4/1/1. Extraction of the silica gel with methanol is followed by evaporation to dryness in vacuo to result in the product.

Yield: 4 mg (41% of theory)

LC-MS (method 18): Rₜ=1.11 min.

MS (EI): In/Z=514 (M+H)⁺

### Example 9

### Isopropyl(8S,11S,14S)-14-amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxylate dihydrochloride

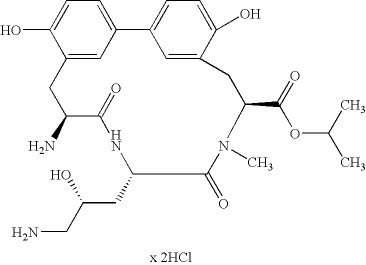

Preparation takes place in analogy to Example 5 from 10 mg (0.02 mmol) of the compound from Example 19A and 1 ml of isopropanol with 10 drops of 4M dioxane/hydrogen chloride solution.

Yield: 1.2 mg (11% of theory)

LC-MS (method 21): Rₜ=1.10 min.

MS (EI): m/z 528 (M+H)⁺

### Example 10

### 2-Aminoethyl-(8S,11S,14S)-14-amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxylate trihydrochloride

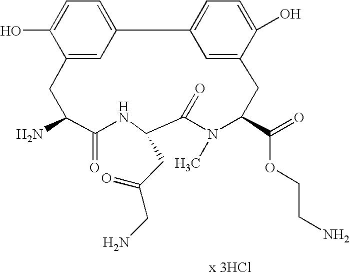

Preparation takes place in analogy to Example 4 from 4 mg of the compound from Example 53A with 1 ml of 4M dioxane/hydrogen chloride solution, reaction time: 60 minutes.

Yield: 3 mg (97% of theory)

HPLC (method 25): Rₜ=3.0 min.

MS (EI): m/z=528 (M+H)⁺

### Example 11

### Isobutyl(8S,11S,14S)-14-amino-11-(3-aminopropyl)-5,17-dihydroxy-9-methyl-10,13-dioxo-9,12-diazatricyclo[14.3.1.1^{2,6}]henicosa-1(20),2(21),3,5,16,18-hexaene-8-carboxylate dihydrochloride

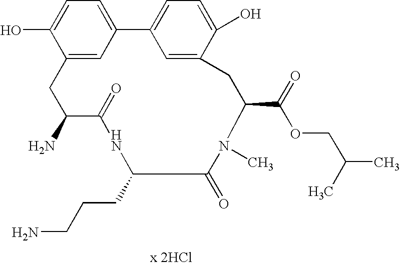

Preparation takes place in analogy to Example 5 from 5 mg. (0.01 mmol) of the compound from Example 28A and 2 ml of isobutanol with 10 drops of 4M dioxane/hydrogen chloride solution.

Yield: 5 mg (89% of theory)

LC-MS (method 21): Rₜ=1.14 min.

MS (EI): m/z 526 (M+H)⁺

B. Assessment of the Physiological Activity

The in vitro effect of the compounds of the invention can be shown in the following assays:

In Vitro Transcription-Translation with E. coli Extracts

An S30 extract is prepared by harvesting logarithmically growing Escherichia coli MRE 600 (M. Müller; University Freiburg), washing and employing them as described for the in vitro transcription-translation assay (Müller, M. and Blobel, G. Proc Natl Acad Sci USA (1984) 81, pp. 7421-7425).

1 μl of cAMP (11.25 mg/ml) are additionally added per 50 μl of reaction mix to the reaction mix for the in vitro transcription-translation assay. The assay mixture amounts to 105 μl, with 5 μl of the substance to be tested being introduced in 5% strength DMSO. 1 μg/100 pt of mixture of the plasmid pBESTLuc (Promega, Germany) are used as transcription template. After incubation at 30° C. for 60 min, 50 pt of luciferin solution (20 mM tricine, 2.67 mM MgSO4, 0.1 mM EDTA, 33.3 mM DTT pH 7.8, 270 μM CoA, 470 μM luciferin, 530 μM ATP) are added, and the resulting bioluminescence is measured in a luminometer for 1 minute. The IC₅₀ is indicated by the concentration of an inhibitor which leads to 50% inhibition of the translation of firefly luciferase.

In Vitro Transcription-Translation with S. aureus Extracts

Construction of an S. aureus Luciferase Reporter Plasmid

A reporter plasmid which can be used in an in vitro transcription-translation assay for S. aureus is constructed by using the plasmid pBESTluc (Promega Corporation, USA). The E. coli tac promoter present in this plasmid in front of the firefly luciferase is replaced by the capA1 promoter with appropriate Shine-Dalgarno sequence from S. aureus. The primers CAPFor 5′-CGGCCAAGCTTACTCGGAT-CCAGAGTTTGCAAAATATACAGGGGATTATATATAATGGAAAACAAGAA AGGAAAATAGGAGGTTTATATGGAAGACGCCA-3′ and CAPRev 5′-GTCATCGTCGGGAAGACCTG-3′ are used for this. The primer CAPFor contains the capA1 promoter, the ribosome binding site and the 5′ region of the luciferase gene. After PCR using pBESTluc as template it is possible to isolate a PCR product which contains the firefly luciferase gene with the fused capA1 promoter. This is, after restriction with ClaI and HindIII, ligated into the vector pBESTluc which has likewise been digested with ClaI and HindIII. The resulting plasmid p1a is able to replicate in E. coli and be used as template in the S. aureus in vitro transcription-translation assay.

Preparation of S30 Extracts from S. aureus

Six liters of BHI medium are inoculated with a 250 ml overnight culture of an S. aureus strain and allowed to grow at 37° C. until the OD600 nm is 2-4. The cells are harvested by centrifugation and washed in 500 ml of cold buffer A (10 mM Tris acetate, pH 8.0, 14 mM Mg acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM Mg acetate, 1 mM DTT, 50 mM KCl). 1.5 ml portions of lysostaphin (0.8 mg/ml) in buffer B are each introduced into 3 precooled centrifuge cups and each mixed with 33 ml of the cell suspension. The samples are incubated at 37° C., shaking occasionally, for 45 to 60 min, before 150 μl of a 0.5 M DTT solution are added. The lyzed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volume of buffer C (670 mM Tris acetate, pH 8.0, 20 mM Mg acetate, 7 mM Na₃ phosphenolpyruvate, 7 mM DTT, 5.5 mM ATP, 70 μM amino acids (complete from Promega), 75 μg of pyruvate kinase (Sigma, Germany)/ml are added to the upper ⅔ of the supernatant. The samples are incubated at 37° C. for 30 min. The supernatants are dialyzed against 2 l of dialysis buffer (10 mM Tris acetate, pH 8.0, 14 mM Mg acetate, 1 mM DTT, 60 mM K acetate) in a dialysis tube with a 3500 Da cut-off with one buffer change at 4° C. overnight. The dialysate is concentrated to a protein concentration of about 10 mg/ml by covering the dialysis tube with cold PEG 8000 powder (Sigma, Germany) at 4° C. The S30 extracts can be stored in aliquots at −70° C.

Determination of the IC₅₀ in the S. aureus In Vitro Transcription-Translation Assay

Inhibition of protein biosynthesis of the compounds can be shown in an in vitro transcription-translation assay. The assay is based on the cell-free transcription and translation of firefly luciferase using the reporter plasmid p1a as template and cell-free S30 extracts obtained from S. aureus. The activity of the resulting luciferase can be detected by luminescence measurement.

The amount of S30 extract or plasmid p1a to be employed must be tested anew for each preparation in order to ensure an optimal concentration in the assay. 3 μl of the substance to be tested, dissolved in 5% DMSO, are introduced into an MTP. Then 10 μl of a suitably concentrated plasmid solution p1a are added. Then 46 μl of a mixture of 23 μl of premix (500 mM K acetate, 87.5 mM Tris acetate, pH 8.0; 67.5 mM ammonium acetate, 5 mM DTT, 50 μg of folic acid/ml, 87.5 mg of PEG 8000/ml, 5 mM ATP, 1.25 mM each NTP, 20 μM each amino acid, 50 mM PEP (Na₃ salt), 2.5 mM cAMP, 250 μg of each E. coli tRNA/ml) and 23 μl of a suitable amount of S. aureus S30 extract are added and mixed. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are, and the resulting bioluminescence is measured in a luminometer for 1 min. The IC₅₀ is indicated as the concentration of an inhibitor which leads to 50% inhibition of the translation of firefly luciferase.

Determination of the Minimum Inhibitory Concentration (MIC):

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtiter plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the exception of phenylalanine; [H.-P. Kroll; unpublished]) with addition of 0.4% BH broth (test medium). In the case of Enterococcus faecalis ICB 27159, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of Enterococci) in fresh test medium, and incubated 1:1 with dilutions of the test substances (1:2 dilution steps) in test medium (150 μl final volume). The cultures are incubated at 37° C. for 18-24 hours; Enterococci in the presence of 5% CO₂.

The lowest substance concentration in each case at which bacterial growth was no longer visible is defined as the MIC. The MIC values in μM of some compounds of the invention for a series of test microbes are listed by way of example in the table below. The compounds show a graded antibacterial effect against most of the test microbes.

TABLE A

| | | | | | | IC₅₀ | IC₅₀ | IC₅₀ |
|---|---|---|---|---|---|---|---|---|
| | MIC | MIC | MIC | MIC | MIC | E. coli | S. aureus | S. aureus |
| Ex. | S. aureus | S. aureus | S. aureus | E. faecalis | B. catarrhalis | MRE600 | 133 | RN4220 |
| No. | 133 | RN4220 | 25701 | ICB 27159 | M3 | Translation | Translation | Translation |
| 1 | 3.13 | 0.4 | 12.5 | 1.56 | 1.56 | 0.5 | 0.5-3.0 | 1.7 |
| 2 | 0.78 | | | | 6.25 | 0.2 | 2.4-4.3 | |
| 10 | 3.1 | | | | | | 0.7 | |

All concentration data in μM.

Systemic Infection with S. aureus 133

The suitability of the compounds of the invention for treating bacterial infections can be shown in various animal models. For this purpose, the animals are generally infected with a suitable virulent microbe and then treated with the compound to be tested, which is in a formulation which is adopted to the particular therapy model. The suitability of the compounds of the invention can be demonstrated specifically for the treatment of bacterial infections in a mouse sepsis model after infection with S. aureus.

For this purpose, S. aureus 133 cells are cultured overnight in BH broth (Oxoid, Germany). The overnight culture is diluted 1:100 in fresh BH broth and expanded for 3 hours. The bacteria which are in the logarithmic phase of growth are centrifuged and washed 2× with buffered physiological saline solution. A cell suspension in saline solution with an extinction of 50 units is then adjusted in a photometer (Dr. Lange LP 2W). After a dilution step (1:15), this suspension is mixed 1:1 with a 10% strength mucine suspension. 0.2 ml of this infection solution is administered i.p. per 20 g of mouse. This corresponds to a cell count of about 1-2×10E6 microbes/mouse. The i.v. therapy takes place 30 minutes after the infection. Female CFW1 mice are used for the infection test. The survival of the animals is recorded for 6 days. The animal model is adjusted so that untreated animals die within 24 h after the infection.

C. Exemplary Embodiments of Pharmaceutical Compositions

The compounds of the invention can be converted into pharmaceutical preparations in the following ways:

Tablet:

Composition:

100 mg of the compound of Example 2, 50 mg of lactose (monohydrate), 50 mg of corn starch (native), 10 mg of polyvinylpyrolidone (PVP 25) (from BASF, Ludwigshafen, Germany) and 2 mg of magnesium stearate.

Tablet weight 212 mg, diameter 8 mm, radius of curvature 12 mm.

Production:

A mixture of active ingredient, lactose and starch is granulated with a 5% strength solution (m/m) of the PVP in water. The granules are dried and then mixed with the magnesium stearate for 5 min. This mixture is compressed with a conventional tablet press (see above for format of the tablet). A compressive force of 15 kN is used as guideline for the compression.

Suspension which can be Administered Orally:

Composition:

1000 mg of the compound of Example 2, 1000 mg of ethanol (96%), 400 mg of Rhodigel (xanthan gum from FMC, Pennsylvania, USA) and 99 g of water.

10 ml of oral suspension correspond to a single dose of 100 mg of the compound of the invention.

Production:

The Rhodigel is suspended in ethanol, and the active ingredient is added to the suspension. The water is added with stirring. The mixture is stirred for about 6 h until the swelling of the Rhodigel is complete.

1. A compound of the formula 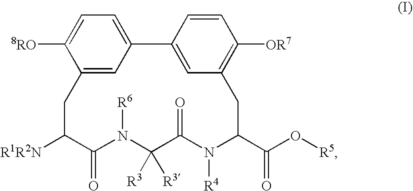
in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
where R¹ apart from hydrogen may be substituted by 0, 1, 2, or 3 substitutents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, and carboxyl,
R² is hydrogen or alkyl,
where alkyl may be substituted by 0, 1, 2, or 3 substituents R²⁻¹, where the substituents R²⁻¹ are selected independently of one another from the group consisting of halogen, amino, alkylamino and dialkylamino, or
R¹ and R² together with the nitrogen atom to which they are bonded form a heterocycle which may be substituted by 0, 1, or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2, or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
and in which one or more free amino groups in the side group of the amino acid may be substituted by alkyl, alkenyl, alkynyl, cycloalkyl, aryl, heteroaryl, heterocyclyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is alkyl, cycloalkyl, aryl, heteroaryl, heterocyclyl or a hydroxy function-linked amino acid residue, where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
in which alkylamino and dialkylamino may be substituted by 0, 1, or 2 substituents R⁵⁻², where the substituents R⁵⁻² are selected independently of one another from the group consisting of hydroxy, amino, alkoxy, alkylamino and dialkylamino,
R⁶ is hydrogen, C₁-C₆-alkyl or C₃-C₈-cycloalkyl,
R⁷ is hydrogen, C₁-C₆-alkyl, alkylcarbonyl or C₃-C₈-cycloalkyl,
R⁸ is hydrogen or C₁-C₆-alkyl,
and one of their salts, their solvates and the solvates of their salts.

2. The compound as claimed in claim 1, characterized in that it corresponds to the formula 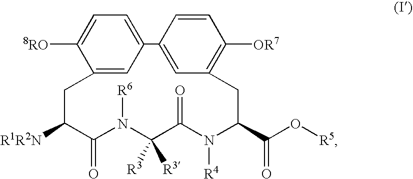
in which R¹ to R⁸ have the same meanings as in formula (I).

3. The compound as claimed in claim 1 or 2, characterized in that
R¹ is hydrogen, alkyl or alkylcarbonyl,
R² is hydrogen,
R³ is alkyl or the side group of an amino acid, in which alkyl may be substituted by 0, 1, 2, or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
and in which one or more free amino groups in the side group of the amino acid may be substituted by alkyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is alkyl, cycloalkyl, aryl, heteroaryl or heterocyclyl, where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
in which alkylamino and dialkylamino may be substituted by 0, 1, or 2 substituents R⁵⁻², where the substituents R⁵⁻² are selected independently of one another from the group consisting of hydroxy, amino, alkoxy, alkylamino and dialkylamino,
R⁶ is hydrogen,
R⁷ is hydrogen, C₁-C₆-alkyl, alkylcarbonyl or C₃-C₈-cycloalkyl, and
R⁸ is hydrogen.

4. The compound as claimed in claim 3, characterized in that
R¹ is hydrogen,
R² is hydrogen,
R³ is aminocarbonylmethyl, 3-aminoprop-1-yl, 2-hydroxy-3-aminoprop-1-yl, 1-hydroxy-3-aminoprop-1-yl, 3-guanidinoprop-1-yl, 2-aminocarbonylethyl, 2-hydroxycarbonylethyl, 4-aminobut-1-yl, hydroxymethyl, 2-hydroxyethyl, 2-aminoethyl, 4-amino-3-hydroxybut-1yl or (1-piperidin-3-yl)methyl,
R^{3′} is hydrogen,
R⁴ is hydrogen, methyl, ethyl, isopropyl or cyclopropyl,
R⁵ is alkyl or C₃-C₆-cycloalkyl, where R⁵ may be substituted by 0, 1, 2 or 3 substituents R⁵⁻¹, where the substituents R⁵⁻¹ are selected independently of one another from the group consisting of alkyl, amino, alkylamino, dialkylamino, cycloalkyl, hydroxy, alkoxy, carboxyl, alkoxycarbonyl, aminocarbonyl, alkylaminocarbonyl and dialkylaminocarbonyl,
in which alkylamino and dialkylamino may be substituted by 0, 1, or 2 substituents R⁵⁻², where the substituents R⁵⁻² are selected independently of one another from the group consisting of hydroxyl and amino,
R⁶ is hydrogen,
R⁷ is hydrogen, and
R⁸ is hydrogen.

5. The compound as claimed in claim 4, characterized in that
R¹ is hydrogen,
R² is hydrogen,
R³ is 3-aminoprop-1-yl or 2-hydroxy-3-aminoprop-1-yl,
R^{3′} is hydrogen,
R⁴ is hydrogen or methyl,
R⁵ is C₁-C₄-alkyl where alkyl may be substituted by 0, 1 or 2 substituents independently of one another selected from the group consisting of amino, hydroxyl and carboxyl,
R⁶ is hydrogen,
R⁷ is hydrogen, and
R⁸ is hydrogen.

6. The compound as claimed in claim 1 or 2, characterized in that
R¹ is hydrogen.

7. The compound as claimed in claim 1 or 2, characterized in that R² is hydrogen.

8. The compound as claimed in claim 1 or 2, characterized in that R³ is 3-aminoprop-1-yl or 2-hydroxy-3-aminoprop-1-yl.

9. The compound as claimed in claim 1 or 2, characterized in that R^{3′} is hydrogen.

10. The compound as claimed in claim 1 or 2, characterized in that R⁴ is hydrogen or methyl.

11. The compound as claimed in claim 1 or 2, characterized in tht R⁵ is C₁-C₄-alkyl, where alkyl may be substituted by 0, 1 or 2 substituents independently of one another selected from the group consisting of amino, hydroxyl and carboxyl.

12. The compound as claimed in claim 1 or 2, characterized in that R⁶ is hydrogen.

13. The compound as claimed in claim 1 or 2, characterized in that R⁷ is hydrogen.

14. The compound as claimed in claim 1 or 2, characterized in that R⁸ is hydrogen.

15. A process for preparing a compound of the formula (I) as claimed in claim 1, characterized in that a compound of the formula 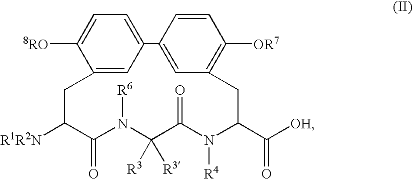
in which
R¹ to R⁴ and R⁶ to R⁸ have the meaning indicated in claim 1,
is reacted with a compound of the formula
HO—R⁵ (III),
in which
R⁵ has the meaning indicated in claim 1.

16. (canceled)

17. A medicament comprising at least one compound as claimed in claim 1 or 2 in combination with at least one pharmaceutically suitable, pharmaceutically acceptable carrier or other excipients.

18. (canceled)

19. A medicament as claimed in claim 17 for the treatment and/or prophylaxis of bacterial infections.

20. A method for controlling bacterial infections in humans and animals by administration of an antibacterially effective amount of at least one compound as claimed in claim 1 or 2.
